SEQUENCE LISTING (51 sequences)

SEQ ID NO: 1 - DNA, 21 bp, synthetic construct
  source 1..21: mol_type = other DNA
caataacgctggatacatctg

SEQ ID NO: 2 - DNA, 19 bp, synthetic construct
  source 1..19: mol_type = other DNA
tgaatctgctgtcatccat

SEQ ID NO: 3 - DNA, 41 bp, synthetic construct
  source 1..41: mol_type = other DNA
atggatgacagcagattcacagtgtatttacttgagaggag

SEQ ID NO: 4 - DNA, 22 bp, synthetic construct
  source 1..22: mol_type = other DNA
catttccccctttgatttttag

SEQ ID NO: 5 - DNA, 43 bp, synthetic construct
  source 1..43: mol_type = other DNA
tctaaaaatcaaagggggaaatgaaggagtcagtccaagagat

SEQ ID NO: 6 - DNA, 23 bp, synthetic construct
  source 1..23: mol_type = other DNA
agaaatagagaacttacatattc

SEQ ID NO: 7 - DNA, 60 bp, synthetic construct
  source 1..60: mol_type = other DNA
gaatatgtaagttctctatttctaaaggaggaataaaaaatgtccagttgtaaaaccact

SEQ ID NO: 8 - DNA, 19 bp, synthetic construct
  source 1..19: mol_type = other DNA
tcaagcaagactatcaagc

SEQ ID NO: 9 - DNA, 40 bp, synthetic construct
  source 1..40: mol_type = other DNA
cgcttgatagtcttgcttgagaattgaacctctaccacaa

SEQ ID NO: 10 - DNA, 19 bp, synthetic construct
  source 1..19: mol_type = other DNA
gggtgatgtctaaggttga

SEQ ID NO: 11 - DNA, 21 bp, synthetic construct
  source 1..21: mol_type = other DNA
ttgagaggagacagtagactt

SEQ ID NO: 12 - DNA, 19 bp, synthetic construct
  source 1..19: mol_type = other DNA
gcatctaacttgacccaga

SEQ ID NO: 13 - DNA, 19 bp, synthetic construct
  source 1..19: mol_type = other DNA
tcctatgagcaatggcaat

SEQ ID NO: 14 - DNA, 20 bp, synthetic construct
  source 1..20: mol_type = other DNA
attgctagtctcgtttcctt

SEQ ID NO: 15 - DNA, 18 bp, synthetic construct
  source 1..18: mol_type = other DNA
cgtttccgccattctttg

SEQ ID NO: 16 - DNA, 20 bp, synthetic construct
  source 1..20: mol_type = other DNA
cttcatataacgcagcatca

SEQ ID NO: 17 - DNA, 40 bp, synthetic construct
  source 1..40: mol_type = other DNA
accatcaacgcaaccataaactgaaccaacgacgctaaga

SEQ ID NO: 18 - DNA, 21 bp, synthetic construct
  source 1..21: mol_type = other DNA
agatgttagcgacagtaatag

SEQ ID NO: 19 - DNA, 40 bp, synthetic construct
  source 1..40: mol_type = other DNA
tctattactgtcgctaacatctcggtgaaggtctgaatca

SEQ ID NO: 20 - DNA, 19 bp, synthetic construct
  source 1..19: mol_type = other DNA
aggaaggctgataggtcat

SEQ ID NO: 21 - DNA, 40 bp, synthetic construct
  source 1..40: mol_type = other DNA
catgacctatcagccttccttcttcaactaaagcacccat

SEQ ID NO: 22 - DNA, 20 bp, synthetic construct
  source 1..20: mol_type = other DNA
ttattcattcagttttcgtg

SEQ ID NO: 23 - DNA, 41 bp, synthetic construct
  source 1..41: mol_type = other DNA
gcacgaaaactgaatgaataacggctaacatagaaggagat

SEQ ID NO: 24 - DNA, 20 bp, synthetic construct
  source 1..20: mol_type = other DNA
gctacatcatacacggtaac

SEQ ID NO: 25 - DNA, 18 bp, synthetic construct
  source 1..18: mol_type = other DNA
atctctggtgcgatgaca

SEQ ID NO: 26 - DNA, 20 bp, synthetic construct
  source 1..20: mol_type = other DNA
gacgataaacccaaacacaa

SEQ ID NO: 27 - DNA, 41 bp, synthetic construct
  source 1..41: mol_type = other DNA
accatcaacgcaaccataaaccgcaactctttcattctctt

SEQ ID NO: 28 - DNA, 21 bp, synthetic construct
  source 1..21: mol_type = other DNA
tggagttagccacagtaatag

SEQ ID NO: 29 - DNA, 40 bp, synthetic construct
  source 1..40: mol_type = other DNA
tctattactgtggctaactccatcattggcacgaagaagg

SEQ ID NO: 30 - DNA, 18 bp, synthetic construct
  source 1..18: mol_type = other DNA
gattctgcggacggtgta

SEQ ID NO: 31 - DNA, 39 bp, synthetic construct
  source 1..39: mol_type = other DNA
gtacaccgtccgcagaatctcttcaactaaagcacccat

SEQ ID NO: 32 - DNA, 39 bp, synthetic construct
  source 1..39: mol_type = other DNA
gcacgaaaactgaatgaataaggttgattggagcctgag

SEQ ID NO: 33 - DNA, 18 bp, synthetic construct
  source 1..18: mol_type = other DNA
cgtcctgcgtattgagaa

SEQ ID NO: 34 - DNA, 19 bp, synthetic construct
  source 1..19: mol_type = other DNA
tcttcctgatgctggtgaa

SEQ ID NO: 35 - DNA, 1309 bp, Bacillus subtilis
  source 1..1309: mol_type = genomic DNA
caataacgctggatacatctgcccctttgataaaatccttgttcatcccctctacttttttaacaaaaagatcctctgcccgaagctctgacacgtgcttctccttctcaatggcagcggcatatcctgttgaactacatgcactccacacgatggcagccgcaaagaacattttcactttgcttttcatgatttcatccccccaagacttagcaagcgttttcattctatagaaaaaagaatccgcccatatcgagcggagcatcagcctaatgtgtgtttacgacaattctcacttcatacttttccatcgtcaggtcgcctgacaatatgtctcctgtcattatgtccttcacactctgatcaaacgtgaccagctgtttttcttccgtgaaattcatgacaaaaatataatcattgtcctgatcctgcctcgcttgtacggagacgccttttccgtgccgaaccggaaaaactggagagagagacaggtctgtgatcagaccctcatagaaatcacgctgaaattgatcctccaaacgcgcgccgataaaatacgccttgccctgctgatactcatggcttgtgaccgctggcgtgcgcgcataaaaatcttcttgatacaccgcttccactgaagctgtctttacatcaatcacggttgcataatccttcatttcatatatttggctgcggtagctgacagcgtttcgatccttcggatacagggtgtccgtttcaagaggctcaactccaaatatagcttgaagatccggatgccatccgcctgtgtatgttaagtcatgctcattcacaaccccgctgatatacgtcatgactaaggtgccgccgtcagccgtaaacgcttttaaacgggaaacggtgtcctcgctgattaaatacagcatcgggacgatcagcagtttatatggtgaaaagtcttgttctttcgtgatgacgtcgacagggatatcgtgttcccagaatgtgcggtaatgctgctgaagcgtttgcggataacgttttgtcgccttcgcaaacccctgagcatcctcgagcgcccaatgattttcccagtcatataaaatcgcggtttgagccggcctcttcgttccgacaacttcggacagccgttccaatgtctcgcctaccttggccacttcttgaaagacgcggttcttcgggctattgtcatgatccacaaccgctccgtgtaatttttctgatgacccccgtgatttgcggtattggaaatagagaacgctgtccgagccgtgggcaatcatttgcatggatgacagcagattca

SEQ ID NO: 36 - DNA, 426 bp, synthetic construct
  source 1..426: mol_type = other DNA
cagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgacaaatggtccaaactagtgatatctaaaaatcaaagggggaaatg

SEQ ID NO: 37 - DNA, 1976 bp, synthetic construct
  source 1..1976: mol_type = other DNA
gaattgaacctctaccacaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaaggagctaaagaggtccctagactctagacccggggatctctgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgcagcaatggcaagaacgttgctctagagcggccgcatcgattcacagtggcaatctcccccgtattcgtttgaaatgtgccacattaacagcgccgggtgatgtccgtatcgttctgctaataagcggttgatgtgccgtgttttttctcggtagactttagatgtgaggcagtggttgtgccttccgccgtgcagctgtttgacgcgggaggcattgacgcgcaaaacttccggataggtttgcgacagccaggccggacgggctccgctcggcgttgctaatatgacccggccgcctatactgtgaatccgctcaaaaatatcatccagccattcaaattgatatacgccctcctccggctcaagtgcgctccatgcaaaaatgccgacagaaaacgtattcgtatgagaaagcttcatcagtttgatatcgtcagctaaaatatcgggccgatccagccactgatcggggttgtagtctcccccatggagcataaattttgcttttgttacgtgcgttttttcaaccttagacatcaccc

SEQ ID NO: 38 - DNA, 631 bp, Saccharomyces cerevisiae
  source 1..631: mol_type = genomic DNA
aaggagtcagtccaagagatcatccagcaactcatccacagtgtcgatttacagtcttccaagttccagctggccattgtgtgcacgatgttcaatcctatcttttggaacatcgttgcaagaatggaataccacaagcattctctcaccaagatgtgtggtggggccagaaagggctgttacatgttggcggcgaccatattttcgctaggtatcgtcagagacatggtgtacgagtctgcattgcgtgaacagcctacgtgttctctgatcacgggcgagaactggaccaagctgggtgtggctctctttggtttggggcaagtgcttgttttgagttccatgtacaagctgggtatcacagggacgtacttgggtgactatttcggcatcctgatggatgagagagtcaccggcttccccttcaacgtttccaacaaccccatgtaccagggttccactttgtccttcttgggcatagccctttacaaaggaaagcctgcggggctggttgtttctgccgtagtttacttcatgtacaagatcgctcttcgttgggaagaaccttttactgccatgatctacgctaaccgtgataaggccaaaaagaatatgtaagttctctatttct

SEQ ID NO: 39 - DNA, 2626 bp, Saccharomyces cerevisiae
  source 1..2626: mol_type = genomic DNA
aaaggaggaataaaaaatgtccagttgtaaaaccactttgtctgaaatggttggttctgtgacaaaagataggggcactatcaacgtcgaagccagaacacgttcgagtaatgtaactttcaaaccacctgtaacccacgatatggtgcgttcgcttttcgatccaactttgaagaaatctctgctggaaaaatgcatcgcgctagctattatatcgaattttttcatttgttattgggtcttccaaaggtttggcttacagtttaccaaatacttttttctggtacagtatttattttggagaattgcttataatttaggtattggactggttctgcattaccagtcacattatgaaacactaacgaattgtgctaaaactcatgcgattttcagcaaaataccacagaataaagatgctaattcgaatttctcaacaaattccaattccttttcagaaaaattttggaattttattaggaagttctgtcaatatgagattaggtctaaaatgccaaaggagtatgatttatttgcctatccagaagagatcaacgtctggttgatatttcgccagtttgtcgatttaatcttgatgcaagattttgtgacttacattatttacgtttacctttctattccatatagctgggttcaaatcttcaactggagatctttgctaggtgttattttgattttatttaacatctgggtcaagttagatgcgcatcgcgtggttaaagattatgcttggtactggggtgatttcttctttttggaagaatctgaattgatttttgatggtgtcttcaacatctctccacatccaatgtattctattggttatttgggttattatggcctatcattgatttgtaatgactacaaggttcttctggtgtccgtatttggacattattctcaatttttgtttctgaaatacgtcgaaaatcctcatattgaaagaacgtatggtgatggcactgattccgattctcaaatgaatagcagaattgatgacttgatatcaaaagagaattacgattattcaagacctctaattaacatgggtctttcttttaacaacttcaataagctaagatttactgactatttcacaattggtaccgtagcggcacttatgttaggaacaatcatgaatgctaggtttatcaatttaaattacctctttattaccgtttttgtaacaaaactggtttcatggttatttatctcgacaatattgtacaagcaatctcagtctaaatggttcacgagattgtttttggagaatggttacacacaagtttattcctatgagcaatggcaatttatttacaattattatttggtattaacctacacacttatgataattcatacagggcttcaaatttggagtaatttttccaacataaacaacagtcaattaatttttggtttaattcttgtggctttacaaacatggtgtgataaggaaacgagactagcaatctctgattttggttggttttatggtgattttttcttgagcaactatatctcaactagaaagctgacttcccaaggtatctacagatatttgaatcacccagaagcggtattaggcgttgttggagtttggggtactgtattgatgacgaattttgccgttacaaatattatcttggctgttttatggacattgacaaattttattcttgtcaagtttattgaaacacctcatgttaataagatatatggtaaaactaaacgagttagtggtgttggaaaaactttactaggcttgaaacctctaagacaagtttcagatattgtcaatagaattgaaaatattataatcaaatcattagttgatgaaagcaagaactccaatggtggggcagaacttttgcccaaaaattatcaagataacaaagaatggaatattttaattcaggaagcaatggatagcgttgcaacaagattgagtccatactgtgaattgaaaattgaaaacgagcaggtagaaaccaattttgtcttgcctactccagtaactttgaattggaaaatgcccattgaactttataacggagatgattggattggtttgtataaagtcattgatacaagggcagatcgtgaaaaaacaagagttggatcaggtggtcattggagtgcgacttctaaagattcatatatgaaccatggattaagacataaagaatctgtaacagaaataaaggcgactgaaaagtacgttcaaggtaaagtgacattcgacacgtctttgctttattttgaaaatggtatctacgaatttaggtatcattctggtaactcccataaggtgttattgatttctacaccatttgaaatttcattgccggtactaaataccacgacacctgaactgtttgagaaggatttgactgagtttttgacgaaagtcaacgttttgaaggatggtaaatttcgtccattaggaaataagttcttcgggatggacagcttgaaacaattgattaagaactcaattggcgtcgaactttcgtctgagtatatgaggagagtcaatggtgacgctcacgttatttcgcatcgcgcttgggatataaaacaaacgcttgatagtcttgcttga

SEQ ID NO: 40 - DNA, 6968 bp, synthetic construct
  source 1..6968: mol_type = other DNA
caataacgctggatacatctgcccctttgataaaatccttgttcatcccctctacttttttaacaaaaagatcctctgcccgaagctctgacacgtgcttctccttctcaatggcagcggcatatcctgttgaactacatgcactccacacgatggcagccgcaaagaacattttcactttgcttttcatgatttcatccccccaagacttagcaagcgttttcattctatagaaaaaagaatccgcccatatcgagcggagcatcagcctaatgtgtgtttacgacaattctcacttcatacttttccatcgtcaggtcgcctgacaatatgtctcctgtcattatgtccttcacactctgatcaaacgtgaccagctgtttttcttccgtgaaattcatgacaaaaatataatcattgtcctgatcctgcctcgcttgtacggagacgccttttccgtgccgaaccggaaaaactggagagagagacaggtctgtgatcagaccctcatagaaatcacgctgaaattgatcctccaaacgcgcgccgataaaatacgccttgccctgctgatactcatggcttgtgaccgctggcgtgcgcgcataaaaatcttcttgatacaccgcttccactgaagctgtctttacatcaatcacggttgcataatccttcatttcatatatttggctgcggtagctgacagcgtttcgatccttcggatacagggtgtccgtttcaagaggctcaactccaaatatagcttgaagatccggatgccatccgcctgtgtatgttaagtcatgctcattcacaaccccgctgatatacgtcatgactaaggtgccgccgtcagccgtaaacgcttttaaacgggaaacggtgtcctcgctgattaaatacagcatcgggacgatcagcagtttatatggtgaaaagtcttgttctttcgtgatgacgtcgacagggatatcgtgttcccagaatgtgcggtaatgctgctgaagcgtttgcggataacgttttgtcgccttcgcaaacccctgagcatcctcgagcgcccaatgattttcccagtcatataaaatcgcggtttgagccggcctcttcgttccgacaacttcggacagccgttccaatgtctcgcctaccttggccacttcttgaaagacgcggttcttcgggctattgtcatgatccacaaccgctccgtgtaatttttctgatgacccccgtgatttgcggtattggaaatagagaacgctgtccgagccgtgggcaatcatttgcatggatgacagcagattcacagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgacaaatggtccaaactagtgatatctaaaaatcaaagggggaaatgaaggagtcagtccaagagatcatccagcaactcatccacagtgtcgatttacagtcttccaagttccagctggccattgtgtgcacgatgttcaatcctatcttttggaacatcgttgcaagaatggaataccacaagcattctctcaccaagatgtgtggtggggccagaaagggctgttacatgttggcggcgaccatattttcgctaggtatcgtcagagacatggtgtacgagtctgcattgcgtgaacagcctacgtgttctctgatcacgggcgagaactggaccaagctgggtgtggctctctttggtttggggcaagtgcttgttttgagttccatgtacaagctgggtatcacagggacgtacttgggtgactatttcggcatcctgatggatgagagagtcaccggcttccccttcaacgtttccaacaaccccatgtaccagggttccactttgtccttcttgggcatagccctttacaaaggaaagcctgcggggctggttgtttctgccgtagtttacttcatgtacaagatcgctcttcgttgggaagaaccttttactgccatgatctacgctaaccgtgataaggccaaaaagaatatgtaagttctctatttctaaaggaggaataaaaaatgtccagttgtaaaaccactttgtctgaaatggttggttctgtgacaaaagataggggcactatcaacgtcgaagccagaacacgttcgagtaatgtaactttcaaaccacctgtaacccacgatatggtgcgttcgcttttcgatccaactttgaagaaatctctgctggaaaaatgcatcgcgctagctattatatcgaattttttcatttgttattgggtcttccaaaggtttggcttacagtttaccaaatacttttttctggtacagtatttattttggagaattgcttataatttaggtattggactggttctgcattaccagtcacattatgaaacactaacgaattgtgctaaaactcatgcgattttcagcaaaataccacagaataaagatgctaattcgaatttctcaacaaattccaattccttttcagaaaaattttggaattttattaggaagttctgtcaatatgagattaggtctaaaatgccaaaggagtatgatttatttgcctatccagaagagatcaacgtctggttgatatttcgccagtttgtcgatttaatcttgatgcaagattttgtgacttacattatttacgtttacctttctattccatatagctgggttcaaatcttcaactggagatctttgctaggtgttattttgattttatttaacatctgggtcaagttagatgcgcatcgcgtggttaaagattatgcttggtactggggtgatttcttctttttggaagaatctgaattgatttttgatggtgtcttcaacatctctccacatccaatgtattctattggttatttgggttattatggcctatcattgatttgtaatgactacaaggttcttctggtgtccgtatttggacattattctcaatttttgtttctgaaatacgtcgaaaatcctcatattgaaagaacgtatggtgatggcactgattccgattctcaaatgaatagcagaattgatgacttgatatcaaaagagaattacgattattcaagacctctaattaacatgggtctttcttttaacaacttcaataagctaagatttactgactatttcacaattggtaccgtagcggcacttatgttaggaacaatcatgaatgctaggtttatcaatttaaattacctctttattaccgtttttgtaacaaaactggtttcatggttatttatctcgacaatattgtacaagcaatctcagtctaaatggttcacgagattgtttttggagaatggttacacacaagtttattcctatgagcaatggcaatttatttacaattattatttggtattaacctacacacttatgataattcatacagggcttcaaatttggagtaatttttccaacataaacaacagtcaattaatttttggtttaattcttgtggctttacaaacatggtgtgataaggaaacgagactagcaatctctgattttggttggttttatggtgattttttcttgagcaactatatctcaactagaaagctgacttcccaaggtatctacagatatttgaatcacccagaagcggtattaggcgttgttggagtttggggtactgtattgatgacgaattttgccgttacaaatattatcttggctgttttatggacattgacaaattttattcttgtcaagtttattgaaacacctcatgttaataagatatatggtaaaactaaacgagttagtggtgttggaaaaactttactaggcttgaaacctctaagacaagtttcagatattgtcaatagaattgaaaatattataatcaaatcattagttgatgaaagcaagaactccaatggtggggcagaacttttgcccaaaaattatcaagataacaaagaatggaatattttaattcaggaagcaatggatagcgttgcaacaagattgagtccatactgtgaattgaaaattgaaaacgagcaggtagaaaccaattttgtcttgcctactccagtaactttgaattggaaaatgcccattgaactttataacggagatgattggattggtttgtataaagtcattgatacaagggcagatcgtgaaaaaacaagagttggatcaggtggtcattggagtgcgacttctaaagattcatatatgaaccatggattaagacataaagaatctgtaacagaaataaaggcgactgaaaagtacgttcaaggtaaagtgacattcgacacgtctttgctttattttgaaaatggtatctacgaatttaggtatcattctggtaactcccataaggtgttattgatttctacaccatttgaaatttcattgccggtactaaataccacgacacctgaactgtttgagaaggatttgactgagtttttgacgaaagtcaacgttttgaaggatggtaaatttcgtccattaggaaataagttcttcgggatggacagcttgaaacaattgattaagaactcaattggcgtcgaactttcgtctgagtatatgaggagagtcaatggtgacgctcacgttatttcgcatcgcgcttgggatataaaacaaacgcttgatagtcttgcttgagaattgaacctctaccacaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaaggagctaaagaggtccctagactctagacccggggatctctgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgcagcaatggcaagaacgttgctctagagcggccgcatcgattcacagtggcaatctcccccgtattcgtttgaaatgtgccacattaacagcgccgggtgatgtccgtatcgttctgctaataagcggttgatgtgccgtgttttttctcggtagactttagatgtgaggcagtggttgtgccttccgccgtgcagctgtttgacgcgggaggcattgacgcgcaaaacttccggataggtttgcgacagccaggccggacgggctccgctcggcgttgctaatatgacccggccgcctatactgtgaatccgctcaaaaatatcatccagccattcaaattgatatacgccctcctccggctcaagtgcgctccatgcaaaaatgccgacagaaaacgtattcgtatgagaaagcttcatcagtttgatatcgtcagctaaaatatcgggccgatccagccactgatcggggttgtagtctcccccatggagcataaattttgcttttgttacgtgcgttttttcaaccttagacatcaccc

SEQ ID NO: 41 - DNA, 1302 bp, Bacillus subtilis
  source 1..1302: mol_type = genomic DNA
cttcatataacgcagcatcaatttgttgtgccagatccttgctgaatgaaaaaggcacaatttggtcgtccttagaagcaatgacagcacggtgcttcgcggactcgatgattttttgatgatcaaacgatccctgtgtaaattcatctaacatctgtaaagtgggcaatgatttggcaaagccggaaacaagtatgattccgccaagctgttttcgtaattgaagatgttccaaaaatctcaaaatggcggggcaacctaagctgtgcgcaaccaaatacgtattttcatgtaaagtgtgctgatataaagagagtgtatccagccaatcctcaagtcttggctggagcggattaggcatgtttaaaatgtctgcttggaccccatctgcgagcagacgttttttcagccaagggaaccaatgatttgttgaggaggctctgtagccatgaatgatatagacttgttttgtcattatttcctcctaagaaatattttcattcagtataaaattcctctcattgggaaacaagtacgcacttttttgtaagtagctttacatatgaggccctccttgtgccattccccacttacacatctcatctatgaggggatacaaagatcgccctctgtcagataacgaatactctactttcagtggggtgtgagaaacctcagaccgatgaataaggccgtcagtttgcaattccttaagctgtgcgcttaacattttatgggtgatgaagggcatagcccttttcagctcgccgtatctcatcgttttttcacaccctaactcatagagaattctcattttccatttgccccctataagagataaggtgtattcaaatggggcttgtttactgcggcacaggctattcattgtaatcaaacctccttttttaagtatcatatcagaataaattaggattttcggtgttgaagtttgagggcgattcacgatctgaagtgaaaggggccgcttgctcaacaggcggcacggtacatgaatgttctaagtcaaagagggttaccttactgttagttactttatttaaaaaaacgcatgtttttagctgaaaatatctattatggtatatttaaatataaagaaatatttgagaaatgaggaatggtatgaatacacccaaaaaacataagaaatttaaagcgaaaatgattctccaaatcatcatggttatcattggaggaatcatagcggcatatggactagaaacagtgctcattccaaacagtgtatctgatggaggggtgacgggtcttagcgtcgttggttca

SEQ ID NO: 42 - DNA, 1005 bp, Bacillus subtilis
  source 1..1005: mol_type = genomic DNA
cggtgaaggtctgaatcacagaaataccaagggcgtcaaaacccttggtattttttctggttattagattttatcttcagctttacgtgctgcttcgagcttatcaatcctgtctttatatttttctccccagtccttcatgtgcaggataacaggttctaatgttcttccaaactcagttaaagagtattcgacttttggagggacctgtttataaacctcgcggtgaatcacgccatcccgttccaattctcttaattgtagcgtgagcatacgctgggtaatttttgggtatagttttcgaaactcattaaacctctttttaccgtcaatgagatgataaagaagaatgcctttccacttacctccgatgatatctaatgttgtctccaccggacatccttcaaagtcatcagaagaataatcaatgccgagtttacgatttctcaagctgttcactcctaatagtatactttttgatactatatacattcaatgtgcgtactttttatattttaatatacatcatataatgatgtcacatctaaggaatgaaaggaatggatcatatgaaaacactcgtactcgttgtacatccgaatatagaatcctctcgtatcaataaaaagtggaaagaagccgttttaagtgaaccagatgtaactgtccatgatctttatgaaaaatatcgcgatcaaccaattgatgtggaatttgaacaacagcagctcctggcccatgaccgtatcgtttttcagtttccattatactggtacagcagcccaccgcttttaaaacagtggtttgatgaagtgtttacgtttggctgggctcatggtcccggcggaaataaattgaaggggaaagagtgggtaactgccatgtccatcggttcacctgaacactcttatcaagccggcggatataacttgttttcgataagcgagctgacaaaaccgttccaagcatctgcccatttagtaggcatgacctatcagccttcct

SEQ ID NO: 43 - DNA, 2069 bp, synthetic construct
  source 1..2069: mol_type = other DNA
tcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaaggcattttctgtcaatgttttcttacaaagaacgctgtgatatactgaaatttgtccgtatacattttggaggaatggatatgttaccaaaatacgcgcaagtaaaagaagaaatcagttcttggattaatcaaggcaaaatactgcccgatcaaaaaatccctaccgaaaacgaattaatgcagcaattcggcgtcagccggcataccatccgcaaagcgatcggagacctcgtatcacaaggtctgctgtacagcgtgcaaggcggaggcacctttgtcgcttcacgctctgctaagtcagcgctgcattccaataaaacgatcggtgttttgacaacttacatatcagactatattttcccgagcatcatcagaggaatcgagtcctatttaagcgagcaggggtattctatgcttttgacaagcacaaacaacaacccggacaatgaaagaagaggcttagaaaacctgctgtcccagcatattgacggactcatcgtagaaccgacaaaaagcgcccttcaaaccccaaacatcggctattatctgaacttggagaaaaacggcattccttttgcgatgattaacgcgtcatatgccgagcttgccgcgccaagttttaccttggatgatgtgaaaggcgggatgatggcggcggagcatttgctttctctcggccacacgcatatgatgggtatttttaaagctgatgacacacaaggcgtgaaacggatgaacggatttatacaggcgcaccgggagcgtgagttgtttccttctccggatatgatcgtgacatttacaacggaagaaaaagaatcaaaacttctggagaaagtaaaagccacactggagaaaaacagcaagcacatgccgacagccattctttgttataacgatgaaattgcgctgaaggtgattgatatgctgagggagatggatcttaaagtgccggaggatatgtctattgtcgggtacgatgattcacatttcgcccaaatctcagaagtgaaactaacctctgtcaaacatccgaaatcagtgcttggaaaagcagccgccaaatatgtcattgactgcttagagcataaaaagccgaagcaagaggatgtcatatttgagcctgagttgatcattcgccagtccgcacgaaaactgaatgaataa

SEQ ID NO: 44 - DNA, 514 bp, Bacillus subtilis
  source 1..514: mol_type = genomic DNA
cggctaacatagaaggagataccctgttaattaccgttgtcggcgggattatccttggattcggaatggggctggcgctgcgtaatggcggcgcattagatggaattgacatgcttgctgtgctgctatcccgaaaactgccattcggaacgagcgatctcattctattcttaaacctatttgtgtttatttttgtttccacagtatttggcttacaaggggcattgctttcagtcattgcttactacattgcatccaaagtgattcatgttgtggaggaaggcttaagcggctctaaaacctttcaaattattacaacccaacctgaattaatggtagagacaatacgtgatcaattaggccgaagcgccacatataaagaagcttacgggggtttttcccacgagaaatttaaagaaatcacatgtgtcatcaaccgtttagaagaaaccaaactaaaggaaattattaatgatattgataaaactgcctttgttaccgtgtatgatgtagc

SEQ ID NO: 45 - DNA, 3329 bp, synthetic construct
  source 1..3329: mol_type = other DNA
gtttatggttgcgttgatggttaaatctcaaaaaggtgttgacattcaaaacaaaatatggtataatgcccccaaaatcgcaaaaaagtgttgacaacttaactcagatctggtataatagaaacacagaatagtcttttaagtaagtctactctgaatttttttaaaaggagagggtaaagaatggcaaacccaacaacagggaagtcctcgattagggctaagctttctaactcatcgctatcaaacctatttaaaaaaaataaaaataaaagacagcgtgaggaaacggaagagcaggacaatgaggataaggatgagagtaagaaccaggatgaaaataaggacacacagctcactccccgcaagcgtcgccggttgacgaaggagtttgaagagaaggaggctcgttacaccaacgagttgcccaaggaactgcgcaagtatcgtcctaaaggtttcagattcaatttgcctccaacggatagacccatcaggatatatgcagatggtgtttttgatcttttccatcttggccacatgaagcaactggaacagtgtaagaaggctttccccaatgtaacactgatagttggtgtgcctagcgacaaaatcactcacaaactaaaaggtttgactgtgctgaccgataagcagcgttgtgaaactttaacgcactgcagatgggttgacgaagtcgtgcccaacgctccctggtgtgtcaccccagaatttctactagaacacaagattgactacgtggcacatgacgatattccttacgttagcgccgacagcgacgatatctacaagccaataaaggagatgggaaaattcttgactacccaaagaaccaatggtgtctctacaagtgatattatcacaaagatcatcagagattatgacaaatatttgatgagaaactttgcaaggggtgctaccagacaggagctgaacgtttcttggttgaagaaaaacgaactggagttcaaaaaacacatcaatgaattcaggtcatatttcaagaaaaaccagacaaatttgaataacgcctccagagacttgtacttcgaagtccgtgaaatcttgctaaagaaaacgttgggcaaaaaactctactccaagttaataggcaatgaattaaagaaacaaaatcaacgacaaagaaaacagaattttttggatgatccgtttactaggaagctaatcagggaggcctctccggctacagagtttgccaacgaatttacgggcgaaaactctaccgctaaatcaccggatgacaatggaaatcttttcagtcaggaagatgatgaagacacaaattctaacaacacgaatacaaattcagattcagattcaaacactaactcaacgcctcccagtgaagatgacgacgacaacgacaggttaactttggaaaacctaacacagaagaagaaacaatcagcgaactaagtaaggtcaaaaggaggaattaaaaatggtacaagaatcacgtccagggagtgtaagaagttactcggtcggttaccaagcaaggtccagatcgagttctcaaagaagacattcgttaacacgccaacgttcctcgcaaagactgattagaaccatcagtatcgagtctgatgtgtctaatattactgacgatgacgatttgagagctgtcaatgagggagtagcgggtgtgcaactggacgtctctgaaaccgcaaataagggaccaagaagagcatcagcaactgatgtcacagatagtttgggttcgacttcgtcggaatatattgagattccctttgttaaggaaacattggatgcaagtttaccttcggattatctgaagcaggacatattaaatctcattcagagtttgaagatatccaaatggtataacaacaagaaaatccaaccggtagcacaagatatgaacttagtcaagatctctggtgcgatgacaaacgcaattttcaaagttgaataccctaagttaccatcgttgctattgagaatatacggaccgaatattgataatatcattgacagggaatatgaattgcagattttggctaggctttcattgaaaaatataggtccttccctttacggctgttttgtaaacggtagatttgagcagtttctggagaattctaagactttaacaaaagacgacattagaaactggaagaactctcaaaggattgcaaggagaatgaaggagttacatgtaggtgttcctctcttgagttcagaaaggaagaacgggtcggcttgttggcaaaagattaaccagtggttgcgcacgattgagaaagtcgaccaatgggtgggggatcctaaaaacattgaaaactctttattatgtgagaattggtccaagtttatggatattgtcgatagatatcacaagtggcttatttctcaagaacagggtatagagcaagtcaacaaaaatcttatattctgccataatgatgcccaatacggcaatttacttttcactgctcctgtgatgaacacaccgagcctatacactgcaccttcgtctacatcattgacttcccaatcaagttccttatttccttcgagctccaatgtcattgtagatgatataatcaacccgccaaagcaggagcaaagccaagattccaaattggtcgtcattgattttgaatatgcaggtgccaatcccgccgcatatgatttagcgaatcatctttccgagtggatgtatgattacaacaatgctaaggccccacatcagtgccacgctgatagatatcccgataaagaacaggttttgaatttcttatactcttatgtttcgcatctaaggggtggtgctaaggaacccatagatgaagaggttcaaagactctataagtcaatcattcaatggagacccactgtacaactattttggtcgctctgggccatcctacaaagtggtaaattagagaaaaaagaagcctccactgccatcactagagaagaaattggacccaatggaaaaaaatatatcatcaagactgaacccgaatcccctgaagaagactttgttgaaaatgacgacgagcctgaagctggcgtcagcattgacacgttcgattatatggcttatggtcgtgacaagattgcggtcttttggggcgacctcattggcttaggcataatcaccgaagaagaatgcaaaaatttcagctctttcaagttcctcgatactagttatttgtaatacgtatacgaattccttcaacaaaggccaaggaaataaagcaaataacaataacaccattattttaattttttttctattactgtcgctaacatct

SEQ ID NO: 46 - DNA, 8219 bp, synthetic construct
  source 1..8219: mol_type = other DNA
cttcatataacgcagcatcaatttgttgtgccagatccttgctgaatgaaaaaggcacaatttggtcgtccttagaagcaatgacagcacggtgcttcgcggactcgatgattttttgatgatcaaacgatccctgtgtaaattcatctaacatctgtaaagtgggcaatgatttggcaaagccggaaacaagtatgattccgccaagctgttttcgtaattgaagatgttccaaaaatctcaaaatggcggggcaacctaagctgtgcgcaaccaaatacgtattttcatgtaaagtgtgctgatataaagagagtgtatccagccaatcctcaagtcttggctggagcggattaggcatgtttaaaatgtctgcttggaccccatctgcgagcagacgttttttcagccaagggaaccaatgatttgttgaggaggctctgtagccatgaatgatatagacttgttttgtcattatttcctcctaagaaatattttcattcagtataaaattcctctcattgggaaacaagtacgcacttttttgtaagtagctttacatatgaggccctccttgtgccattccccacttacacatctcatctatgaggggatacaaagatcgccctctgtcagataacgaatactctactttcagtggggtgtgagaaacctcagaccgatgaataaggccgtcagtttgcaattccttaagctgtgcgcttaacattttatgggtgatgaagggcatagcccttttcagctcgccgtatctcatcgttttttcacaccctaactcatagagaattctcattttccatttgccccctataagagataaggtgtattcaaatggggcttgtttactgcggcacaggctattcattgtaatcaaacctccttttttaagtatcatatcagaataaattaggattttcggtgttgaagtttgagggcgattcacgatctgaagtgaaaggggccgcttgctcaacaggcggcacggtacatgaatgttctaagtcaaagagggttaccttactgttagttactttatttaaaaaaacgcatgtttttagctgaaaatatctattatggtatatttaaatataaagaaatatttgagaaatgaggaatggtatgaatacacccaaaaaacataagaaatttaaagcgaaaatgattctccaaatcatcatggttatcattggaggaatcatagcggcatatggactagaaacagtgctcattccaaacagtgtatctgatggaggggtgacgggtcttagcgtcgttggttcagtttatggttgcgttgatggttaaatctcaaaaaggtgttgacattcaaaacaaaatatggtataatgcccccaaaatcgcaaaaaagtgttgacaacttaactcagatctggtataatagaaacacagaatagtcttttaagtaagtctactctgaatttttttaaaaggagagggtaaagaatggcaaacccaacaacagggaagtcctcgattagggctaagctttctaactcatcgctatcaaacctatttaaaaaaaataaaaataaaagacagcgtgaggaaacggaagagcaggacaatgaggataaggatgagagtaagaaccaggatgaaaataaggacacacagctcactccccgcaagcgtcgccggttgacgaaggagtttgaagagaaggaggctcgttacaccaacgagttgcccaaggaactgcgcaagtatcgtcctaaaggtttcagattcaatttgcctccaacggatagacccatcaggatatatgcagatggtgtttttgatcttttccatcttggccacatgaagcaactggaacagtgtaagaaggctttccccaatgtaacactgatagttggtgtgcctagcgacaaaatcactcacaaactaaaaggtttgactgtgctgaccgataagcagcgttgtgaaactttaacgcactgcagatgggttgacgaagtcgtgcccaacgctccctggtgtgtcaccccagaatttctactagaacacaagattgactacgtggcacatgacgatattccttacgttagcgccgacagcgacgatatctacaagccaataaaggagatgggaaaattcttgactacccaaagaaccaatggtgtctctacaagtgatattatcacaaagatcatcagagattatgacaaatatttgatgagaaactttgcaaggggtgctaccagacaggagctgaacgtttcttggttgaagaaaaacgaactggagttcaaaaaacacatcaatgaattcaggtcatatttcaagaaaaaccagacaaatttgaataacgcctccagagacttgtacttcgaagtccgtgaaatcttgctaaagaaaacgttgggcaaaaaactctactccaagttaataggcaatgaattaaagaaacaaaatcaacgacaaagaaaacagaattttttggatgatccgtttactaggaagctaatcagggaggcctctccggctacagagtttgccaacgaatttacgggcgaaaactctaccgctaaatcaccggatgacaatggaaatcttttcagtcaggaagatgatgaagacacaaattctaacaacacgaatacaaattcagattcagattcaaacactaactcaacgcctcccagtgaagatgacgacgacaacgacaggttaactttggaaaacctaacacagaagaagaaacaatcagcgaactaagtaaggtcaaaaggaggaattaaaaatggtacaagaatcacgtccagggagtgtaagaagttactcggtcggttaccaagcaaggtccagatcgagttctcaaagaagacattcgttaacacgccaacgttcctcgcaaagactgattagaaccatcagtatcgagtctgatgtgtctaatattactgacgatgacgatttgagagctgtcaatgagggagtagcgggtgtgcaactggacgtctctgaaaccgcaaataagggaccaagaagagcatcagcaactgatgtcacagatagtttgggttcgacttcgtcggaatatattgagattccctttgttaaggaaacattggatgcaagtttaccttcggattatctgaagcaggacatattaaatctcattcagagtttgaagatatccaaatggtataacaacaagaaaatccaaccggtagcacaagatatgaacttagtcaagatctctggtgcgatgacaaacgcaattttcaaagttgaataccctaagttaccatcgttgctattgagaatatacggaccgaatattgataatatcattgacagggaatatgaattgcagattttggctaggctttcattgaaaaatataggtccttccctttacggctgttttgtaaacggtagatttgagcagtttctggagaattctaagactttaacaaaagacgacattagaaactggaagaactctcaaaggattgcaaggagaatgaaggagttacatgtaggtgttcctctcttgagttcagaaaggaagaacgggtcggcttgttggcaaaagattaaccagtggttgcgcacgattgagaaagtcgaccaatgggtgggggatcctaaaaacattgaaaactctttattatgtgagaattggtccaagtttatggatattgtcgatagatatcacaagtggcttatttctcaagaacagggtatagagcaagtcaacaaaaatcttatattctgccataatgatgcccaatacggcaatttacttttcactgctcctgtgatgaacacaccgagcctatacactgcaccttcgtctacatcattgacttcccaatcaagttccttatttccttcgagctccaatgtcattgtagatgatataatcaacccgccaaagcaggagcaaagccaagattccaaattggtcgtcattgattttgaatatgcaggtgccaatcccgccgcatatgatttagcgaatcatctttccgagtggatgtatgattacaacaatgctaaggccccacatcagtgccacgctgatagatatcccgataaagaacaggttttgaatttcttatactcttatgtttcgcatctaaggggtggtgctaaggaacccatagatgaagaggttcaaagactctataagtcaatcattcaatggagacccactgtacaactattttggtcgctctgggccatcctacaaagtggtaaattagagaaaaaagaagcctccactgccatcactagagaagaaattggacccaatggaaaaaaatatatcatcaagactgaacccgaatcccctgaagaagactttgttgaaaatgacgacgagcctgaagctggcgtcagcattgacacgttcgattatatggcttatggtcgtgacaagattgcggtcttttggggcgacctcattggcttaggcataatcaccgaagaagaatgcaaaaatttcagctctttcaagttcctcgatactagttatttgtaatacgtatacgaattccttcaacaaaggccaaggaaataaagcaaataacaataacaccattattttaattttttttctattactgtcgctaacatctcggtgaaggtctgaatcacagaaataccaagggcgtcaaaacccttggtattttttctggttattagattttatcttcagctttacgtgctgcttcgagcttatcaatcctgtctttatatttttctccccagtccttcatgtgcaggataacaggttctaatgttcttccaaactcagttaaagagtattcgacttttggagggacctgtttataaacctcgcggtgaatcacgccatcccgttccaattctcttaattgtagcgtgagcatacgctgggtaatttttgggtatagttttcgaaactcattaaacctctttttaccgtcaatgagatgataaagaagaatgcctttccacttacctccgatgatatctaatgttgtctccaccggacatccttcaaagtcatcagaagaataatcaatgccgagtttacgatttctcaagctgttcactcctaatagtatactttttgatactatatacattcaatgtgcgtactttttatattttaatatacatcatataatgatgtcacatctaaggaatgaaaggaatggatcatatgaaaacactcgtactcgttgtacatccgaatatagaatcctctcgtatcaataaaaagtggaaagaagccgttttaagtgaaccagatgtaactgtccatgatctttatgaaaaatatcgcgatcaaccaattgatgtggaatttgaacaacagcagctcctggcccatgaccgtatcgtttttcagtttccattatactggtacagcagcccaccgcttttaaaacagtggtttgatgaagtgtttacgtttggctgggctcatggtcccggcggaaataaattgaaggggaaagagtgggtaactgccatgtccatcggttcacctgaacactcttatcaagccggcggatataacttgttttcgataagcgagctgacaaaaccgttccaagcatctgcccatttagtaggcatgacctatcagccttccttcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaaggcattttctgtcaatgttttcttacaaagaacgctgtgatatactgaaatttgtccgtatacattttggaggaatggatatgttaccaaaatacgcgcaagtaaaagaagaaatcagttcttggattaatcaaggcaaaatactgcccgatcaaaaaatccctaccgaaaacgaattaatgcagcaattcggcgtcagccggcataccatccgcaaagcgatcggagacctcgtatcacaaggtctgctgtacagcgtgcaaggcggaggcacctttgtcgcttcacgctctgctaagtcagcgctgcattccaataaaacgatcggtgttttgacaacttacatatcagactatattttcccgagcatcatcagaggaatcgagtcctatttaagcgagcaggggtattctatgcttttgacaagcacaaacaacaacccggacaatgaaagaagaggcttagaaaacctgctgtcccagcatattgacggactcatcgtagaaccgacaaaaagcgcccttcaaaccccaaacatcggctattatctgaacttggagaaaaacggcattccttttgcgatgattaacgcgtcatatgccgagcttgccgcgccaagttttaccttggatgatgtgaaaggcgggatgatggcggcggagcatttgctttctctcggccacacgcatatgatgggtatttttaaagctgatgacacacaaggcgtgaaacggatgaacggatttatacaggcgcaccgggagcgtgagttgtttccttctccggatatgatcgtgacatttacaacggaagaaaaagaatcaaaacttctggagaaagtaaaagccacactggagaaaaacagcaagcacatgccgacagccattctttgttataacgatgaaattgcgctgaaggtgattgatatgctgagggagatggatcttaaagtgccggaggatatgtctattgtcgggtacgatgattcacatttcgcccaaatctcagaagtgaaactaacctctgtcaaacatccgaaatcagtgcttggaaaagcagccgccaaatatgtcattgactgcttagagcataaaaagccgaagcaagaggatgtcatatttgagcctgagttgatcattcgccagtccgcacgaaaactgaatgaataacggctaacatagaaggagataccctgttaattaccgttgtcggcgggattatccttggattcggaatggggctggcgctgcgtaatggcggcgcattagatggaattgacatgcttgctgtgctgctatcccgaaaactgccattcggaacgagcgatctcattctattcttaaacctatttgtgtttatttttgtttccacagtatttggcttacaaggggcattgctttcagtcattgcttactacattgcatccaaagtgattcatgttgtggaggaaggcttaagcggctctaaaacctttcaaattattacaacccaacctgaattaatggtagagacaatacgtgatcaattaggccgaagcgccacatataaagaagcttacgggggtttttcccacgagaaatttaaagaaatcacatgtgtcatcaaccgtttagaagaaaccaaactaaaggaaattattaatgatattgataaaactgcctttgttaccgtgtatgatgtagc

SEQ ID NO: 47 - DNA, 1362 bp, Bacillus subtilis
  source 1..1362: mol_type = genomic DNA
gacgataaacccaaacacaacaccaataagaattgtcagcgccctcttattttcaagcggcatcagctgccagcggttggggcgaagttttctatccaaaaacaaacgaacgatgattgaaatggcaaccagcgttaacacatatcctagagcgtgcttaataatttcttcctgatgctggtgaaaggcggggaacaaatgcaatatgccaatggctagcgaagcactcggaatactgccgatggctaagtattttaccaatttaaagttaatcgttttctgccgccaatgggaggcaacgccaaacagctttgtgatcgagttatatacaagatccgtaccgaccgcgatggaaggattaattccaagcacaattaaaaggggagttaataaggccgctccacccactcccgtcaatccgactaaggctccgacaaacaaacccatcaatatgatgcttacactcatgataaaggcctcctgaaaacccgactatttttatcagattatatatgattaggacgcgttaatcaattaaattttttgaatttttgcacgttagttgcttgaaattttggtatgctcaatatctctattcatttgccctataatgagtgtctggacgaaatcgagaagtactaaaggcgcaaaaaaaagatacctcttaacaggtatcttttttcttgtttacgctacatgcgctgctgatagaaaggaagctcagtgtggcccatttcccgcacatacacgaacaggttgcctttatggtgaatttcatgctccattgcaagctgaagcagcgcgcggcctgtgacttttctgccgaaagcggatgtcagatcaatttctctgtccagctgctcttcagtcagctgttcaaggatagcgaccgttttttctgtatatgtttttgccagaacattgagatctgtttctgtttcttcctgcttgttttgaaatggtgaggcattgccttccttaatgacatttgcgaacaagtggaaagacgtaagaatgtgtttgaccagctcttctgctgacattgatgtttctgccggtttgtagctgtaatggtctttgctgattttttcggcaagctcattggtcacgtttcgatgggataaaaaatggctgacaatttgattggattggcacattatgatctcctccatatgtaaaaataaatggtgcaatgaaatcctatatcaacggttatgaattcacaagtaatatgctgtgggatgaaacaaaatgctatgtcaatcgtatatataacgttcacaaaacatgaaatggagtataaacatgactaacatacctttcatttatcagtacgaagaaaaagagaatgaaagagttgcg

SEQ ID NO: 48 - DNA, 808 bp, Bacillus subtilis
  source 1..808: mol_type = genomic DNA
tcattggcacgaagaagggaatcaccatattgaaaccatggtgaaagacatgacaggagagcaaagaaaaagccagcggggaagctggatggaaagaaacaaagtcggttttcactaaaagaaagcacgggtgtttgaaaaacccgtgcttttttgttgcggttagccgaaattcgacaattgcggttattttgcgttcttctttttcttgtaaatatgataaaatatgacatatctcgggtaattcaaaaggggggattaattgaggatgaagcagacgattccgtcctcttatgtcgggcttaaaattaatgaatggtatactcatatccggcagttccacgtcgctgaagccgaacgggtcaagctcgaagtagaaagagaaattgaggatatggaagaagaccaagatttgctgctgtattattctttaatggagttcaggcaccgtgtcatgctggattacattaagccttttggagaggacacgtcgcagctagagttttcagaattgttagaagacatcgaagggaatcagtacaagctgacagggcttctcgaatattactttaatttttttcgaggaatgtatgaatttaagcagaagatgtttgtcagtgccatgatgtattataaacgggcagaaaagaatcttgccctcgtctcggatgatattgagaaagcagagtttgcttttaaaatggctgagattttttacaatttaaaacaaacctatgtttcgatgagctacgccgttcaggcattagaaacataccaaatgtatgaaacgtacaccgtccgcagaatc

SEQ ID NO: 49 - DNA, 697 bp, Bacillus subtilis
  source 1..697: mol_type = genomic DNA
ggttgattggagcctgagcatgtggcatatatatttgaaaagcttgaaaagaaaacctttgcttacgatgaagatgagaaagaaccggattataccgtccataagtctataagaaacagtgtatacgcgtatcctgaaaagggagttgcttttgccagaattccatattttcaagatgggagcattatgagttttgattgtctgtttgcggttaacgatgaaaagatgcgtgcatttctggagggagtcagaccgcgtctttgggaaaaaagcaagcggaaagtgaccgtgtttacggatggagatggagggacttcaagagagcaggaagccattgtcagagaggttcagcggagtcaagtcatcatgaatccgctattgaaaaaagagatatacagatcaattgatcagttttttcatagtgataaatcgttttatcaaacatatgacatcccttacaagcgcggcattctgttatatggacctcctggaaacggaaagacgacgttagtgaagtcgatcgcaggcagtatcgatgcacctgttgcttattggcaaattactgaatttacgtcgagcgagacaatagaagaagtctttcaggcagcgagacgcctcgctcctgcagttctggtcatcgaggatatagattcgatgccggaagatgtgcggtccttttttctcaatacgcaggacg

SEQ ID NO: 50 - DNA, 3329 bp, synthetic construct
  source 1..3329: mol_type = other DNA
gtttatggttgcgttgatggttaaatctcaaaaaggtgttgacattcaaaacaaaatatggtataatgcccccaaaatcgcaaaaaagtgttgacaacttaactcagatctggtataatagaaacacagaatagtcttttaagtaagtctactctgaatttttttaaaaggagagggtaaagaatggcaaacccaacaacagggaagtcctcgattagggctaagctttctaactcatcgctatcaaacctatttaaaaaaaataaaaataaaagacagcgtgaggaaacggaagagcaggacaatgaggataaggatgagagtaagaaccaggatgaaaataaggacacacagctcactccccgcaagcgtcgccggttgacgaaggagtttgaagagaaggaggctcgttacaccaacgagttgcccaaggaactgcgcaagtatcgtcctaaaggtttcagattcaatttgcctccaacggatagacccatcaggatatatgcagatggtgtttttgatcttttccatcttggccacatgaagcaactggaacagtgtaagaaggctttccccaatgtaacactgatagttggtgtgcctagcgacaaaatcactcacaaactaaaaggtttgactgtgctgaccgataagcagcgttgtgaaactttaacgcactgcagatgggttgacgaagtcgtgcccaacgctccctggtgtgtcaccccagaatttctactagaacacaagattgactacgtggcacatgacgatattccttacgttagcgccgacagcgacgatatctacaagccaataaaggagatgggaaaattcttgactacccaaagaaccaatggtgtctctacaagtgatattatcacaaagatcatcagagattatgacaaatatttgatgagaaactttgcaaggggtgctaccagacaggagctgaacgtttcttggttgaagaaaaacgaactggagttcaaaaaacacatcaatgaattcaggtcatatttcaagaaaaaccagacaaatttgaataacgcctccagagacttgtacttcgaagtccgtgaaatcttgctaaagaaaacgttgggcaaaaaactctactccaagttaataggcaatgaattaaagaaacaaaatcaacgacaaagaaaacagaattttttggatgatccgtttactaggaagctaatcagggaggcctctccggctacagagtttgccaacgaatttacgggcgaaaactctaccgctaaatcaccggatgacaatggaaatcttttcagtcaggaagatgatgaagacacaaattctaacaacacgaatacaaattcagattcagattcaaacactaactcaacgcctcccagtgaagatgacgacgacaacgacaggttaactttggaaaacctaacacagaagaagaaacaatcagcgaactaagtaaggtcaaaaggaggaattaaaaatggtacaagaatcacgtccagggagtgtaagaagttactcggtcggttaccaagcaaggtccagatcgagttctcaaagaagacattcgttaacacgccaacgttcctcgcaaagactgattagaaccatcagtatcgagtctgatgtgtctaatattactgacgatgacgatttgagagctgtcaatgagggagtagcgggtgtgcaactggacgtctctgaaaccgcaaataagggaccaagaagagcatcagcaactgatgtcacagatagtttgggttcgacttcgtcggaatatattgagattccctttgttaaggaaacattggatgcaagtttaccttcggattatctgaagcaggacatattaaatctcattcagagtttgaagatatccaaatggtataacaacaagaaaatccaaccggtagcacaagatatgaacttagtcaagatctctggtgcgatgacaaacgcaattttcaaagttgaataccctaagttaccatcgttgctattgagaatatacggaccgaatattgataatatcattgacagggaatatgaattgcagattttggctaggctttcattgaaaaatataggtccttccctttacggctgttttgtaaacggtagatttgagcagtttctggagaattctaagactttaacaaaagacgacattagaaactggaagaactctcaaaggattgcaaggagaatgaaggagttacatgtaggtgttcctctcttgagttcagaaaggaagaacgggtcggcttgttggcaaaagattaaccagtggttgcgcacgattgagaaagtcgaccaatgggtgggggatcctaaaaacattgaaaactctttattatgtgagaattggtccaagtttatggatattgtcgatagatatcacaagtggcttatttctcaagaacagggtatagagcaagtcaacaaaaatcttatattctgccataatgatgcccaatacggcaatttacttttcactgctcctgtgatgaacacaccgagcctatacactgcaccttcgtctacatcattgacttcccaatcaagttccttatttccttcgagctccaatgtcattgtagatgatataatcaacccgccaaagcaggagcaaagccaagattccaaattggtcgtcattgattttgaatatgcaggtgccaatcccgccgcatatgatttagcgaatcatctttccgagtggatgtatgattacaacaatgctaaggccccacatcagtgccacgctgatagatatcccgataaagaacaggttttgaatttcttatactcttatgtttcgcatctaaggggtggtgctaaggaacccatagatgaagaggttcaaagactctataagtcaatcattcaatggagacccactgtacaactattttggtcgctctgggccatcctacaaagtggtaaattagagaaaaaagaagcctccactgccatcactagagaagaaattggacccaatggaaaaaaatatatcatcaagactgaacccgaatcccctgaagaagactttgttgaaaatgacgacgagcctgaagctggcgtcagcattgacacgttcgattatatggcttatggtcgtgacaagattgcggtcttttggggcgacctcattggcttaggcataatcaccgaagaagaatgcaaaaatttcagctctttcaagttcctcgatactagttatttgtaatacgtatacgaattccttcaacaaaggccaaggaaataaagcaaataacaataacaccattattttaattttttttctattactgtggctaactcca

SEQ ID NO: 51 - DNA, 8099 bp, synthetic construct
  source 1..8099: mol_type = other DNA
tcttcctgatgctggtgaaaggcggggaacaaatgcaatatgccaatggctagcgaagcactcggaatactgccgatggctaagtattttaccaatttaaagttaatcgttttctgccgccaatgggaggcaacgccaaacagctttgtgatcgagttatatacaagatccgtaccgaccgcgatggaaggattaattccaagcacaattaaaaggggagttaataaggccgctccacccactcccgtcaatccgactaaggctccgacaaacaaacccatcaatatgatgcttacactcatgataaaggcctcctgaaaacccgactatttttatcagattatatatgattaggacgcgttaatcaattaaattttttgaatttttgcacgttagttgcttgaaattttggtatgctcaatatctctattcatttgccctataatgagtgtctggacgaaatcgagaagtactaaaggcgcaaaaaaaagatacctcttaacaggtatcttttttcttgtttacgctacatgcgctgctgatagaaaggaagctcagtgtggcccatttcccgcacatacacgaacaggttgcctttatggtgaatttcatgctccattgcaagctgaagcagcgcgcggcctgtgacttttctgccgaaagcggatgtcagatcaatttctctgtccagctgctcttcagtcagctgttcaaggatagcgaccgttttttctgtatatgtttttgccagaacattgagatctgtttctgtttcttcctgcttgttttgaaatggtgaggcattgccttccttaatgacatttgcgaacaagtggaaagacgtaagaatgtgtttgaccagctcttctgctgacattgatgtttctgccggtttgtagctgtaatggtctttgctgattttttcggcaagctcattggtcacgtttcgatgggataaaaaatggctgacaatttgattggattggcacattatgatctcctccatatgtaaaaataaatggtgcaatgaaatcctatatcaacggttatgaattcacaagtaatatgctgtgggatgaaacaaaatgctatgtcaatcgtatatataacgttcacaaaacatgaaatggagtataaacatgactaacatacctttcatttatcagtacgaagaaaaagagaatgaaagagttgcggtttatggttgcgttgatggttaaatctcaaaaaggtgttgacattcaaaacaaaatatggtataatgcccccaaaatcgcaaaaaagtgttgacaacttaactcagatctggtataatagaaacacagaatagtcttttaagtaagtctactctgaatttttttaaaaggagagggtaaagaatggcaaacccaacaacagggaagtcctcgattagggctaagctttctaactcatcgctatcaaacctatttaaaaaaaataaaaataaaagacagcgtgaggaaacggaagagcaggacaatgaggataaggatgagagtaagaaccaggatgaaaataaggacacacagctcactccccgcaagcgtcgccggttgacgaaggagtttgaagagaaggaggctcgttacaccaacgagttgcccaaggaactgcgcaagtatcgtcctaaaggtttcagattcaatttgcctccaacggatagacccatcaggatatatgcagatggtgtttttgatcttttccatcttggccacatgaagcaactggaacagtgtaagaaggctttccccaatgtaacactgatagttggtgtgcctagcgacaaaatcactcacaaactaaaaggtttgactgtgctgaccgataagcagcgttgtgaaactttaacgcactgcagatgggttgacgaagtcgtgcccaacgctccctggtgtgtcaccccagaatttctactagaacacaagattgactacgtggcacatgacgatattccttacgttagcgccgacagcgacgatatctacaagccaataaaggagatgggaaaattcttgactacccaaagaaccaatggtgtctctacaagtgatattatcacaaagatcatcagagattatgacaaatatttgatgagaaactttgcaaggggtgctaccagacaggagctgaacgtttcttggttgaagaaaaacgaactggagttcaaaaaacacatcaatgaattcaggtcatatttcaagaaaaaccagacaaatttgaataacgcctccagagacttgtacttcgaagtccgtgaaatcttgctaaagaaaacgttgggcaaaaaactctactccaagttaataggcaatgaattaaagaaacaaaatcaacgacaaagaaaacagaattttttggatgatccgtttactaggaagctaatcagggaggcctctccggctacagagtttgccaacgaatttacgggcgaaaactctaccgctaaatcaccggatgacaatggaaatcttttcagtcaggaagatgatgaagacacaaattctaacaacacgaatacaaattcagattcagattcaaacactaactcaacgcctcccagtgaagatgacgacgacaacgacaggttaactttggaaaacctaacacagaagaagaaacaatcagcgaactaagtaaggtcaaaaggaggaattaaaaatggtacaagaatcacgtccagggagtgtaagaagttactcggtcggttaccaagcaaggtccagatcgagttctcaaagaagacattcgttaacacgccaacgttcctcgcaaagactgattagaaccatcagtatcgagtctgatgtgtctaatattactgacgatgacgatttgagagctgtcaatgagggagtagcgggtgtgcaactggacgtctctgaaaccgcaaataagggaccaagaagagcatcagcaactgatgtcacagatagtttgggttcgacttcgtcggaatatattgagattccctttgttaaggaaacattggatgcaagtttaccttcggattatctgaagcaggacatattaaatctcattcagagtttgaagatatccaaatggtataacaacaagaaaatccaaccggtagcacaagatatgaacttagtcaagatctctggtgcgatgacaaacgcaattttcaaagttgaataccctaagttaccatcgttgctattgagaatatacggaccgaatattgataatatcattgacagggaatatgaattgcagattttggctaggctttcattgaaaaatataggtccttccctttacggctgttttgtaaacggtagatttgagcagtttctggagaattctaagactttaacaaaagacgacattagaaactggaagaactctcaaaggattgcaaggagaatgaaggagttacatgtaggtgttcctctcttgagttcagaaaggaagaacgggtcggcttgttggcaaaagattaaccagtggttgcgcacgattgagaaagtcgaccaatgggtgggggatcctaaaaacattgaaaactctttattatgtgagaattggtccaagtttatggatattgtcgatagatatcacaagtggcttatttctcaagaacagggtatagagcaagtcaacaaaaatcttatattctgccataatgatgcccaatacggcaatttacttttcactgctcctgtgatgaacacaccgagcctatacactgcaccttcgtctacatcattgacttcccaatcaagttccttatttccttcgagctccaatgtcattgtagatgatataatcaacccgccaaagcaggagcaaagccaagattccaaattggtcgtcattgattttgaatatgcaggtgccaatcccgccgcatatgatttagcgaatcatctttccgagtggatgtatgattacaacaatgctaaggccccacatcagtgccacgctgatagatatcccgataaagaacaggttttgaatttcttatactcttatgtttcgcatctaaggggtggtgctaaggaacccatagatgaagaggttcaaagactctataagtcaatcattcaatggagacccactgtacaactattttggtcgctctgggccatcctacaaagtggtaaattagagaaaaaagaagcctccactgccatcactagagaagaaattggacccaatggaaaaaaatatatcatcaagactgaacccgaatcccctgaagaagactttgttgaaaatgacgacgagcctgaagctggcgtcagcattgacacgttcgattatatggcttatggtcgtgacaagattgcggtcttttggggcgacctcattggcttaggcataatcaccgaagaagaatgcaaaaatttcagctctttcaagttcctcgatactagttatttgtaatacgtatacgaattccttcaacaaaggccaaggaaataaagcaaataacaataacaccattattttaattttttttctattactgtggctaactccatcattggcacgaagaagggaatcaccatattgaaaccatggtgaaagacatgacaggagagcaaagaaaaagccagcggggaagctggatggaaagaaacaaagtcggttttcactaaaagaaagcacgggtgtttgaaaaacccgtgcttttttgttgcggttagccgaaattcgacaattgcggttattttgcgttcttctttttcttgtaaatatgataaaatatgacatatctcgggtaattcaaaaggggggattaattgaggatgaagcagacgattccgtcctcttatgtcgggcttaaaattaatgaatggtatactcatatccggcagttccacgtcgctgaagccgaacgggtcaagctcgaagtagaaagagaaattgaggatatggaagaagaccaagatttgctgctgtattattctttaatggagttcaggcaccgtgtcatgctggattacattaagccttttggagaggacacgtcgcagctagagttttcagaattgttagaagacatcgaagggaatcagtacaagctgacagggcttctcgaatattactttaatttttttcgaggaatgtatgaatttaagcagaagatgtttgtcagtgccatgatgtattataaacgggcagaaaagaatcttgccctcgtctcggatgatattgagaaagcagagtttgcttttaaaatggctgagattttttacaatttaaaacaaacctatgtttcgatgagctacgccgttcaggcattagaaacataccaaatgtatgaaacgtacaccgtccgcagaatctcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaaggcattttctgtcaatgttttcttacaaagaacgctgtgatatactgaaatttgtccgtatacattttggaggaatggatatgttaccaaaatacgcgcaagtaaaagaagaaatcagttcttggattaatcaaggcaaaatactgcccgatcaaaaaatccctaccgaaaacgaattaatgcagcaattcggcgtcagccggcataccatccgcaaagcgatcggagacctcgtatcacaaggtctgctgtacagcgtgcaaggcggaggcacctttgtcgcttcacgctctgctaagtcagcgctgcattccaataaaacgatcggtgttttgacaacttacatatcagactatattttcccgagcatcatcagaggaatcgagtcctatttaagcgagcaggggtattctatgcttttgacaagcacaaacaacaacccggacaatgaaagaagaggcttagaaaacctgctgtcccagcatattgacggactcatcgtagaaccgacaaaaagcgcccttcaaaccccaaacatcggctattatctgaacttggagaaaaacggcattccttttgcgatgattaacgcgtcatatgccgagcttgccgcgccaagttttaccttggatgatgtgaaaggcgggatgatggcggcggagcatttgctttctctcggccacacgcatatgatgggtatttttaaagctgatgacacacaaggcgtgaaacggatgaacggatttatacaggcgcaccgggagcgtgagttgtttccttctccggatatgatcgtgacatttacaacggaagaaaaagaatcaaaacttctggagaaagtaaaagccacactggagaaaaacagcaagcacatgccgacagccattctttgttataacgatgaaattgcgctgaaggtgattgatatgctgagggagatggatcttaaagtgccggaggatatgtctattgtcgggtacgatgattcacatttcgcccaaatctcagaagtgaaactaacctctgtcaaacatccgaaatcagtgcttggaaaagcagccgccaaatatgtcattgactgcttagagcataaaaagccgaagcaagaggatgtcatatttgagcctgagttgatcattcgccagtccgcacgaaaactgaatgaataaggttgattggagcctgagcatgtggcatatatatttgaaaagcttgaaaagaaaacctttgcttacgatgaagatgagaaagaaccggattataccgtccataagtctataagaaacagtgtatacgcgtatcctgaaaagggagttgcttttgccagaattccatattttcaagatgggagcattatgagttttgattgtctgtttgcggttaacgatgaaaagatgcgtgcatttctggagggagtcagaccgcgtctttgggaaaaaagcaagcggaaagtgaccgtgtttacggatggagatggagggacttcaagagagcaggaagccattgtcagagaggttcagcggagtcaagtcatcatgaatccgctattgaaaaaagagatatacagatcaattgatcagttttttcatagtgataaatcgttttatcaaacatatgacatcccttacaagcgcggcattctgttatatggacctcctggaaacggaaagacgacgttagtgaagtcgatcgcaggcagtatcgatgcacctgttgcttattggcaaattactgaatttacgtcgagcgagacaatagaagaagtctttcaggcagcgagacgcctcgctcctgcagttctggtcatcgaggatatagattcgatgccggaagatgtgcggtccttttttctcaatacgcaggacg

(19) United States
(12) Patent Application Publication
(10) Pub. No.: US 2025/0376690 A1
(43) Pub. Date: Dec. 11, 2025

(54) RECOMBINANT ENGINEERED STRAIN FOR DE NOVO SYNTHESIS OF CDP-CHOLINE USING GLUCOSE AS SUBSTRATE AND ITS PREPARATION METHOD AND APPLICATION

(71) Applicants: Shandong Jincheng BIO-PHARMACEUTICAL Co., Ltd., Zibo (CN); Shandong University of Technology, Zibo (CN)

(72) Inventors: Qinyuan Ma, Zibo (CN); Shaomei Yang, Zibo (CN); Hui Wang, Zibo (CN); Xiuzhen Gao, Zibo (CN); Tong Zhang, Zibo (CN); Shouying Fu, Zibo (CN); Ying Li, Zibo (CN); Wei Jin, Zibo (CN); Bin Wang, Zibo (CN); Xu Feng, Zibo (CN)

(21) Appl. No.: 19/186,360

(22) Filed: Apr. 22, 2025

(30) Foreign Application Priority Data
Jun. 11, 2024 (CN) 202410740819.5

(51) Int. Cl. C12N 15/75; C12N 1/20; C12N 9/10; C12P 13/00; C12P 19/30; C12R 1/125

(52) U.S. Cl. CPC C12N 15/75; C12N 1/20; C12N 9/1007; C12P 13/001; C12P 19/305; C12R 2001/125; C12Y 201/01017; C12Y 201/01071

(57) ABSTRACT

A recombinant engineered strain for de novo synthesis of CDP-choline using glucose as a substrate and its preparation method and application are provided. Using BS168N as the starting strain, firstly, the phosphatidylethanolamine N-methyltransferase gene PEM1 and phosphatidylethanolamine/phosphatidyl-N-methylethanolamine N-methyltransferase gene PEM2 from S. cerevisiae are integrated into the genome of the BS168N for induced expression, thereby opening up the synthesis pathway from phosphatidylethanolamine to phosphatidylcholine; subsequently, the CKI and CCT genes of S. cerevisiae are further integrated into the BS168N genome expressing PEM1-PEM2, opening up the synthesis pathway of choline to CDPC, thereby obtaining the recombinant engineered strain. Further, the recombinant engineered strain is subjected to shake flask fermentation to achieve de novo synthesis of CDP-choline using glucose as a substrate. The method of the present disclosure provides a fundamental research and theoretical basis for the construction of efficient cell factories for de novo synthesis of CDP-choline through synthetic biology.

### CROSS-REFERENCE TO RELATED APPLICATIONS

This application claims to the benefit of priority from Chinese Application No. 202410740819.5 with a filing date of Jun. 11, 2024. The content of the aforementioned applications, including any intervening amendments thereto, are incorporated herein by reference.

### SEQUENCE LISTING

The present application contains a sequence listing which was filed electronically in XML format and is hereby incorporated by reference in its entirety. Besides, the XMIL copy is created on Aug. 20, 2025, is named “RECOMBINANT ENGINEERED STRAIN FOR DE NOVO SYNTHESIS OF CDP-CHOLINE USING GLUCOSE AS SUBSTRATE AND ITS PREPARATION METHOD AND APPLICATION-Sequence Listing” and is 94,208 bytes in sizes.

### TECHNICAL FIELD

The present disclosure relates to the technical field of biotechnology, in particular to a recombinant engineered strain for de novo synthesis of CDP-choline using glucose as a substrate and its preparation method and application.

### BACKGROUND

Cytidine diphosphate-choline (CDPC), also known as cytidine 5′-diphosphocholine, is divided into two forms: hydrogen type and sodium type, mainly existing in the form of sodium salt. CDPC is a precursor for the synthesis of phosphatidylcholine, a component of eukaryotic cell membrane, and plays an important role in promoting the repair and regeneration of damaged neurons. Moreover, CDPC is also a precursor substance for synthesizing the neurotransmitter acetylcholine, which has the effects of promoting respiration and energy metabolism of brain cells, improving brain function, and protecting nerves. In addition, CDPC also has anti-apoptotic effects, reducing neuronal apoptosis by increasing the expression of anti-apoptotic factors. Therefore, CDPC has been widely used in the treatment of various neurological diseases, such as consciousness disorders after brain surgery, acute traumatic brain injury, cerebral infarction, senile dementia, vascular dementia, etc.

At present, the synthesis methods of CDPC mainly include chemical synthesis, enzyme catalysis, microbial fermentation, etc.

For example, in 1956, Kennedy achieved the chemical synthesis of CDPC for the first time using cytidine triphosphate (CTP) and phosphatidylcholine as substrates and N, N-dicyclohexylcarbodiimide as a condensation agent. However, this method had problems such as low yield, low purity, high production cost, and the use of toxic reagents. In addition, in 1957, Fencil et al. utilized phosphocholine cytidylyltransferase (CCT) derived from rats to catalyze the reaction between phosphocholine and CTP to generate CDPC, achieving enzymatic synthesis of CDPC for the first time. In 2018, Wang et al. conducted rational mutagenesis on CCT derived from Saccharomyces cerevisiae S288C and obtained salt tolerant CCT mutants that catalyze the generation of CDPC from phosphocholine, with a maximum CDPC yield of 161 mmol/L (82.2 g/L).

Although enzymatic catalysis can achieve high levels of CDPC, it requires the production of enzymes and catalytic reactions to be divided into two stages, which is complex, time-consuming, and not conducive to large-scale production. Therefore, many researchers are currently focusing on using microbial fermentation to produce CDPC.

Professor Chen Ning's research group at Tianjin University of Science and Technology used Escherichia coli K12 MG1655 as the starting strain, and sequentially integrated and expressed the choline kinase gene CKI and the phosphocholine cytidylyltransferase gene CCT from Streptococcus pneumoniae, the cytidine kinase gene udk from Thermus thermophilus, the cytidylate kinase gene cmk and the nucleoside diphosphate kinase gene ndk from Bacillus subtilis. After 12 hours of shake flask fermentation, the recombinant strain were added with choline chloride and cytidine in a final concentration of 100 mM, then after 32 hours of fermentation, the intracellular CDPC production reached 41.3 mg/g cells, and the cell dry weight reached 15.4 g/L. Professor Zhou Xiangshan's research group from East China University of Science and Technology used Pichia pastoris GS115 as the starting strain and expressed the CKI gene and choline transporter gene HNM1 of S. cerevisiae, the endogenous CCT gene and subunit gene sATP6 of ATP synthase, as well as two genes used for knocking out the CMP pathway, were used to increase the concentration of sodium citrate and optimize the addition concentrations of CMP and phosphorylcholine. Using phosphorylcholine as the substrate, the highest CDPC yield was 29.7 g/L. Professor Ma Qinyuan's research group from Shandong University of Technology used wild-type B. subtilis 168 is the starting strain and integrates the expression of the CKI gene and CCT gene derived from S. cerevisiae, the endogenous glycine betaine and arsenate betaine transporter genes opuD, and the knockout of the 5′-nucleotidase gene yfkN were used. Choline chloride and CMP were added to the fermentation medium, and the highest intracellular CDPC production reached 123.8 mg/L (CN116790466B).

However, the microbial fermentation methods used in the above studies all require the addition of precursors such as phosphatidylcholine or choline chloride. The former is expensive, while if the latter is added at a high concentration, high chloride ion (Cl⁻) concentration can interfere with the formation of cell membranes and inhibit the citric acid cycle and glycolysis process. Therefore, if glucose can be directly used as raw material to synthesize CDP-choline from the source, it will greatly reduce production costs.

### SUMMARY

In order to solve the above technical problems, the present disclosure provides a recombinant engineered strain for de novo synthesis of CDP-choline using glucose as a substrate and its preparation method and application.

The present disclosure is first based on the patent CN116790466B, by integrating Pₐᵣₐ-neo into the araR site of the wild-type Bacillus subtilis 168 genome to obtain the engineered strain Bacillus subtilis 168N. Then, using Bacillus subtilis 168N as the starting strain, a recombinant engineered strain capable of de novo synthesis of CDP-choline from glucose as a substrate was prepared.

In fact, Bacillus subtilis can only synthesize phosphatidylethanolamine (PE) from scratch using glucose as a substrate. Therefore, the present disclosure first integrates the phosphatidylethanolamine N-methyltransferase gene gene PEM1 and phosphatidylethanolamine/phosphatidyl-N-methylethanolamine N-methyltransferase gene PEM2 from Saccharomyces cerevisiae into the genome of Bacillus subtilis 168N for induced expression, aiming to open up the synthesis pathway from PE to phosphatidylcholine (PC); subsequently, the CKI and CCT genes of Saccharomyces cerevisiae are further integrated into the genome of Bacillus subtilis expressing PEM2-PEM1, aiming to open up the synthesis pathway of choline to CDPC and obtain the corresponding recombinant engineered strain. Finally, the recombinant engineered strain is subjected to shake flask fermentation to directly synthesize CDPC using glucose as a substrate.

For a recombinant engineered strain for de novo synthesis of CDP-choline using glucose as a substrate provided by the present disclosure, the recombinant engineered strain is obtained by modifying Bacillus subtilis 168N as the chassis as follows:

(1) modification of Bacillus subtilis 168N: combining the phosphatidylethanolamine N-methyltransferase gene PEM1 and phosphatidylethanolamine/phosphatidyl-N-methylethanolamine N-methyltransferase gene PEM2 from Saccharomyces cerevisiae into an artificial operon P_{xyl}-PEM2-PEM1, and integrating the artificial operon P_{xyl}-PEM2-PEM1 into the lacA site of the Bacillus subtilis 168N genome for induced expression.

Preferably, the Bacillus subtilis 168N in step (1) is obtained by integrating Pₐᵣₐ-neo into the araR site of the wild-type Bacillus subtilis 168 genome.

(2) construction of recombinant engineered strain: integrating the artificial operon TP2-CCT-CKI into the genome of Bacillus subtilis expressing P_{xyl}-PEM2-PEM2-PEM1 in step (1), thus constructing a recombinant engineered strain capable of de novo synthesis of CDP-choline using glucose as a substrate.

In the preparation method of recombinant engineered strain mentioned above, more specifically, in (1), the genome of Bacillus subtilis 168N is used as a template, and the fragment U containing the upstream homologous arm is amplified using primers lacA-U1/lacA-U2; taking plasmid pJMP1 as a template, the fragment P containing xylose inducible promoter P_{xyl} and the fragment ED containing erythromycin resistance gene and downstream homologous arm are amplified using primers lacA-P1q/lacA-P2 and lacA-ED1q/lacA-ED2, respectively.

In addition, using the genome of Saccharomyces cerevisiae as a template, the fragment M2 containing the PFA2 gene sequence and M1 containing the PEM1 gene sequence are amplified using primers PEM2-1q/PEM2-2 and PEM1-1q/PEM1-2, respectively; then, using the primer lacA-P1q/PEM1-2, the fragment P, the fragment M2, and the fragment M1 are spliced together to form fragment PM2M1 through overlap-PCR; further, using primer lacA-U1/lacA-ED2 to splice the fragment U, the fragment PM2M1, and the fragment ED into fragment UPM2M1ED; finally, the fragment UPM2M1ED is transformed into competent cells of the receptor bacterium BS168N, and after screening, a PEM2-PEM1 co-expression strain is obtained.

In step (1), the fragment U is shown as SEQ ID No. 35, 1309 bp;
The fragment P is shown as SEQ ID No. 36, 426 bp;
The fragment ED is shown as SEQ ID No. 37, 1976 bp;
The fragment M2 containing the PEM2 gene sequence is shown as SEQ ID No. 38, 631 bp;
The fragment M1 containing the PEM1 gene sequence is shown as SEQ ID No. 39, 2626 bp;
The fragment UPM2M1ED is as shown in SEQ ID No.40, 6968 bp.

In step (2) above, the construction of recombinant strain BS168N/PEM21/ydeO is carried out as follows:
Using the genome of BS168NCm as a template, primers ydeO-U1/ydeO-U2q, ydeO-D1q/ydeO-D2, ydeO-CR1q/ydeO-CR2, and ydeO-D1q/ydeO-G2 are used to amplify the fragment U containing upstream homologous arms, the fragment D containing homologous recombination, the fragment CR containing chloramphenicol resistance gene and arabinose operon repressor gene, and the fragment G containing downstream homologous arms.

Using the BSC1-22 genome as a template, primers TP2-1/ydeO-PCC2 is used to amplify the fragment PCC; then, using primers ydeO-U1/ydeO-PCC2 to splice the fragment U, the fragment PCC, and the fragment D into fragment UPCCD through overlap-PCR; and then using primers ydeO-U1/ydeO-G2 to splice the fragment UPCCD, the fragment CR, and the fragment G into fragment UPCCDCRG; finally, the fragment UPCCDCRG is transformed into competent cells of the receptor bacterium BS168N/PEM21. After screening, a strain co-expressing CCT-CKI at the ydeO site is obtained.

In step (2), the fragment U described in (2) is shown as SEQ ID No. 41, 1302 bp;
The fragment D is shown as SEQ ID No.42, 1005 bp;
The fragment CR is shown as SEQ ID No.43, 2069 bp;
The fragment G is shown as SEQ ID No.44, 514 bp;
The fragment PCC is shown as SEQ ID No.45, 3329 bp;
The fragment UPCCDCRG is shown as SEQ ID No.46, 8219 bp.

Furthermore, in step (2) above, the specific steps for constructing the recombinant strain BS168N/PEM21/yjoB are as follows:

Using the genome of BS168NCm as a template, primers yjoB-U1/yjoB-U2q, yjoB-D1q/yjoB-D2, yjoB-CR1q/ydeO-CR2, yjoB-D1q/yjoB-G2 to amplify the fragment U, the fragment D, the fragment CR, and the fragment G, respectively; using the BSC1-22 genome as a template, primer TP2-1/yjoB-PCC2 is used to amplify the fragment PCC; then, using primers yjoB-U1/yjoB-PCC2, the fragment U, the fragment PCC, and the fragment D are spliced into fragment UPCCD through overlap-PCR; then, primers 2-yjoB-U1/yjoB-G2 are used to splice the fragment UPCCD, the fragment CR, and the fragment G into fragment UPCCDCRG; finally, the fragment UPCCDCRG is transformed into competent cells of the receptor bacterium BS168N/PEM21. After screening, a strain co-expressing CCT-CKI at the yjoB site is obtained, which is constructed as a recombinant engineered strain capable of de novo synthesis of CDP-choline using glucose as a substrate.

Wherein the fragment U described in (2) is shown as SEQ ID No. 47, 1362 bp;

The fragment D is shown as SEQ ID No.48, 808 bp;

The fragment G is shown as SEQ ID No.49, 697 bp;

The fragment PCC is shown as SEQ ID No.50, 3329 bp;

The fragment UPCCDCRG is shown as SEQ ID No.51, 8099 bp.

Furthermore, the application of the recombinant engineered strain provided by the present disclosure in de novo synthesis of CDP-choline using glucose as a substrate is also a key technical content of the present disclosure.

The specific application is: inoculating the single colony of the recombinant engineered strain into a test tube filled with LB liquid culture medium, conducting shake culture, then transferring the inoculum volume of 0.5% to 2% of the fermentation medium volume to a conical flask containing the fermentation medium, conducting shake culture, and then adding xylose with a final concentration of 10 g/L after 2-5 hours of fermentation.

The LB liquid culture medium includes the following components: tryptone 5-15 g/L, yeast extract 1-9 g/L NaCl 5-15 g/L, erythromycin 1-8 μg/mL, neomycin 10-20 μg/mL.

The fermentation medium includes the following components: glucose 20-80 g/L, tryptone 5-15 g/L, yeast extract 1-9 g/L NaCl 5-15 g/L, MgSO₄·7H₂O 0.02-3 g/L, cytidine-5′-phosphate 0.01-4 g/L, and xylose 5-20 g/L.

The fermentation conditions are: pH 6.0-8.0, fermentation temperature 30-45° C., rotation speed 100-250 r/min, and fermentation time 20-50 h.

In addition, the present disclosure provides a method for de novo synthesis of CDP-choline using glucose as a substrate, which includes the following steps:
(1) modification of Bacillus subtilis 168N: combining the phosphatidylethanolamine N-methyltransferase gene PEM1 and phosphatidylethanolamine/phosphatidyl-N-methylethanolamine N-methyltransferase gene PEM2 from Saccharomyces cerevisiae into an artificial operon P_{xyl}-PEM2-PEM1, and integrating the artificial operon P_{xyl}-PEM2-PEM1 into the lacA site of the Bacillus subtilis 168N genome for induced expression;
(2) construction of recombinant engineered strain: integrating the artificial operon TP2-CCT-CKI into the genome of Bacillus subtilis expressing P_{xyl}-PEM2-PEM2-PEM1 in step (1), thus constructing a recombinant engineered strain capable of de novo synthesis of CDP-choline using glucose as the substrate;
(3) inoculating the single colony of the recombinant engineered strain constructed in step (2) into a test tube filled with LB liquid culture medium, conducting shake culture, then transferring the inoculum volume of 0.5% to 2% of the fermentation medium volume to a conical flask containing the fermentation medium, then adding xylose with a final concentration of 10 g/L after 2-5 hours of fermentation, and then conducting shake culture in the conical flask for 20-50 hours to synthesize CDP-choline.

The advantageous effects of the present disclosure are:
(1) The present disclosure provides a recombinant engineered strain for de novo synthesis of CDPC using glucose as a substrate, namely, using the engineered strain Bacillus subtilis 168N as the chassis, integrating two artificial operons P_{xyl}-PEM2-PEM1 and TP2-CCT-CKI composed of PEM1, PEM2, CKI, and CCT from Saccharomyces cerevisiae into the genome of Bacillus subtilis 168N. The synthesis of CDPC was further detected by shake flask fermentation, indicating that the pathway for de novo synthesis of CDPC from glucose has been opened up through the modification of the engineered strain, and that the endogenous lipase in Bacillus subtilis can hydrolyze the ester bond at position 2 of 1,2-diacyl-sn-glycerin-3-phosphocholine to generate 1-acyl-sn-glycero-3-phosphocholine. There does not require further introduction of heterologous genes;
(2) The recombinant engineered strain Bacillus subtilis obtained through modification in the present disclosure can directly synthesize CDPC from glucose as a substrate without the need to add precursor choline chloride during fermentation, greatly reducing production costs. Moreover, the above research results also provide basic research and theoretical basis for the construction of efficient de novo CDPC synthesis Bacillus subtilis cell factories through synthetic biology methods in industry.

### BRIEF DESCRIPTION OF THE DRAWINGS

FIG. 1 is a schematic diagram of the biosynthetic pathway and engineering modification strategy of CDP-choline in Bacillus subtilis provided by the present disclosure;

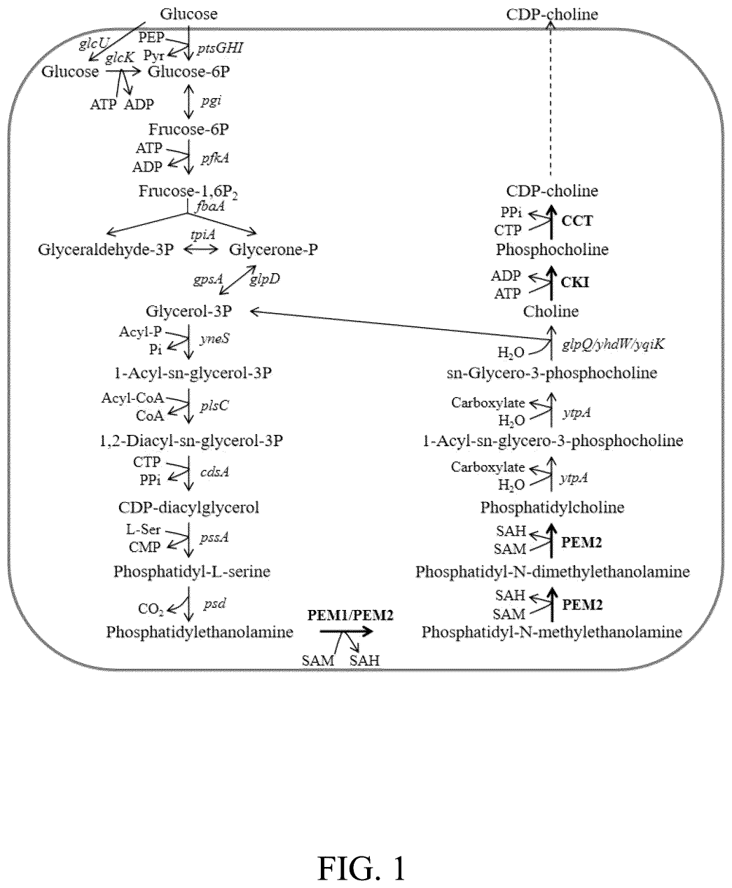

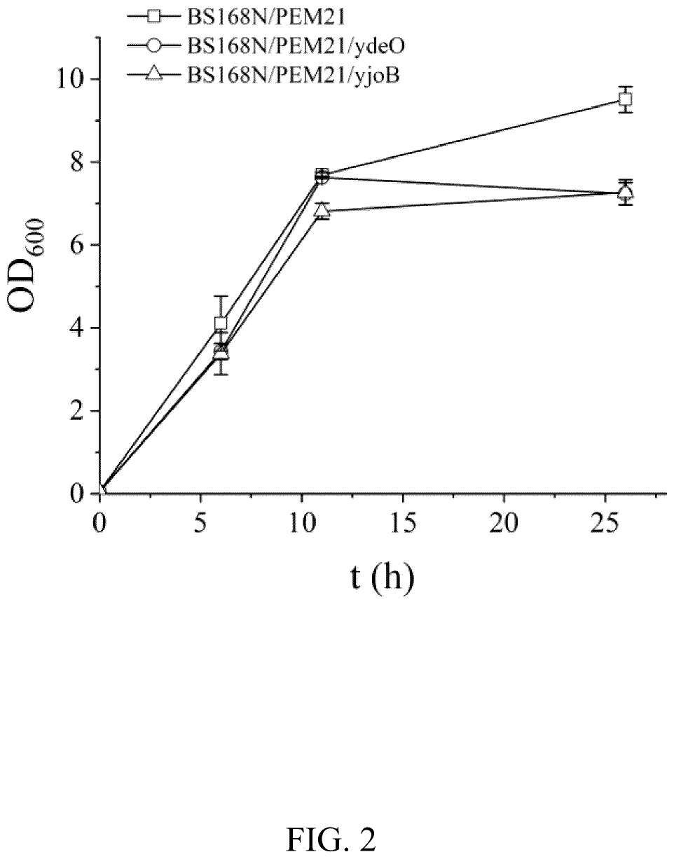

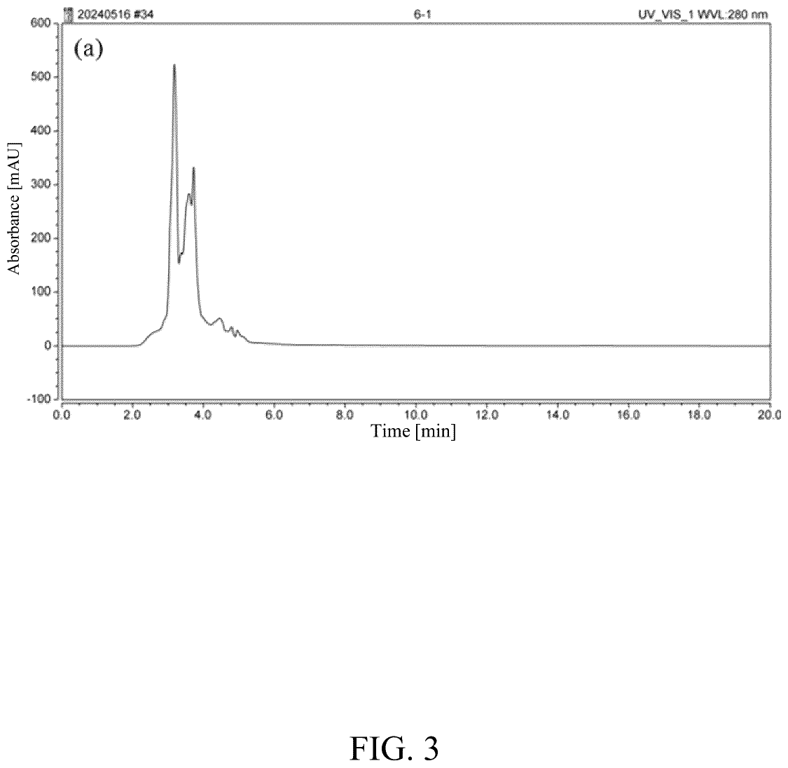

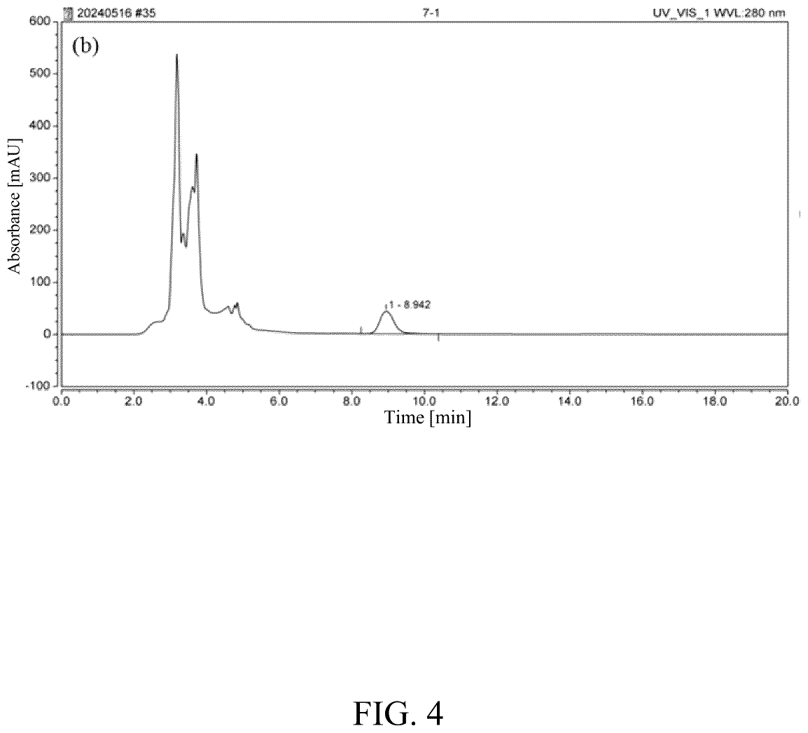

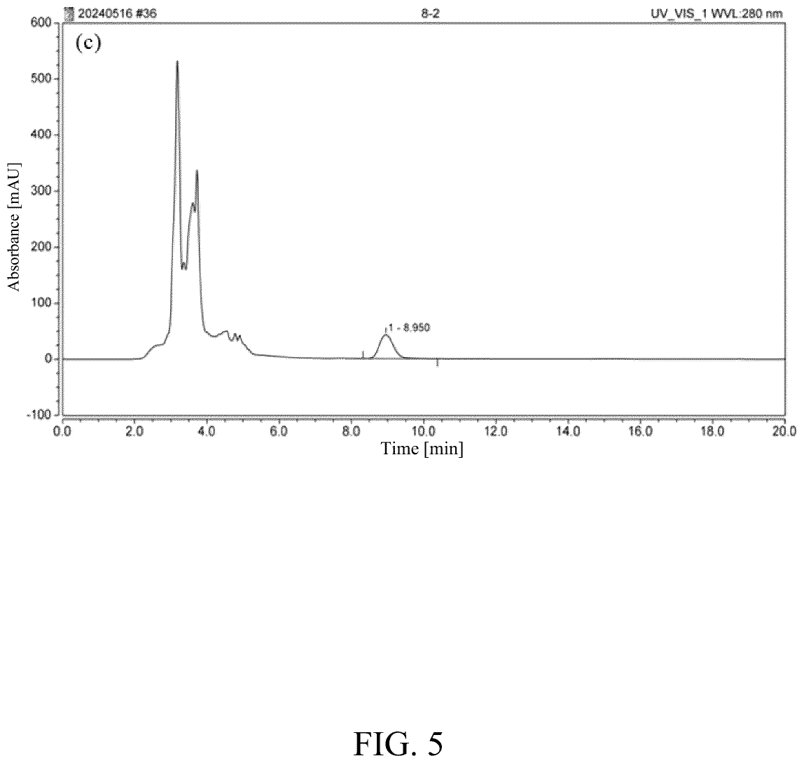

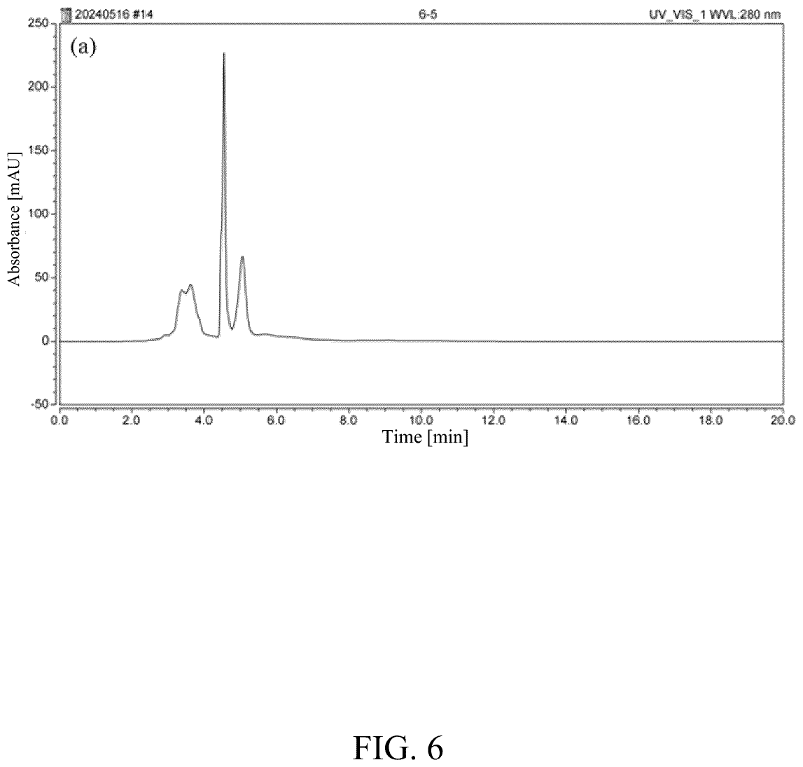

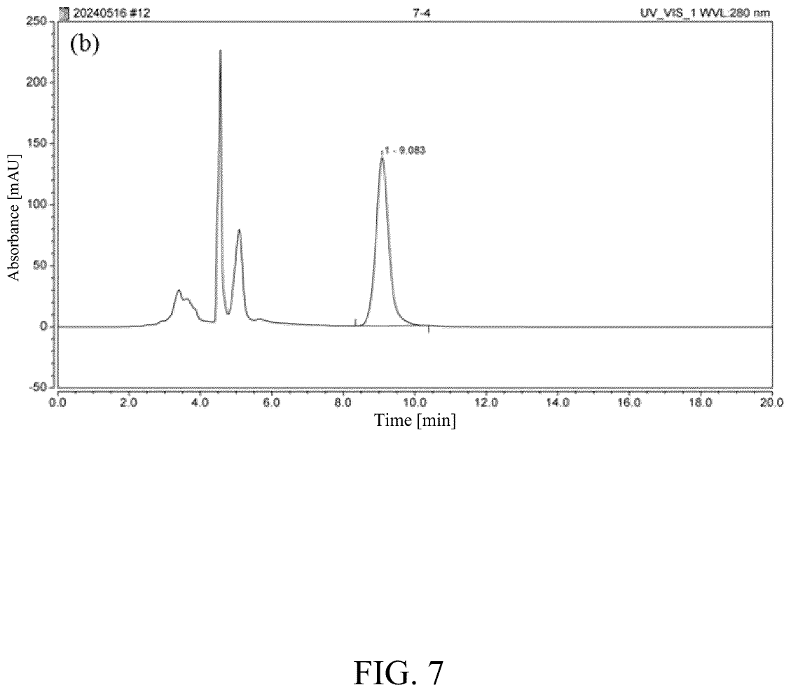

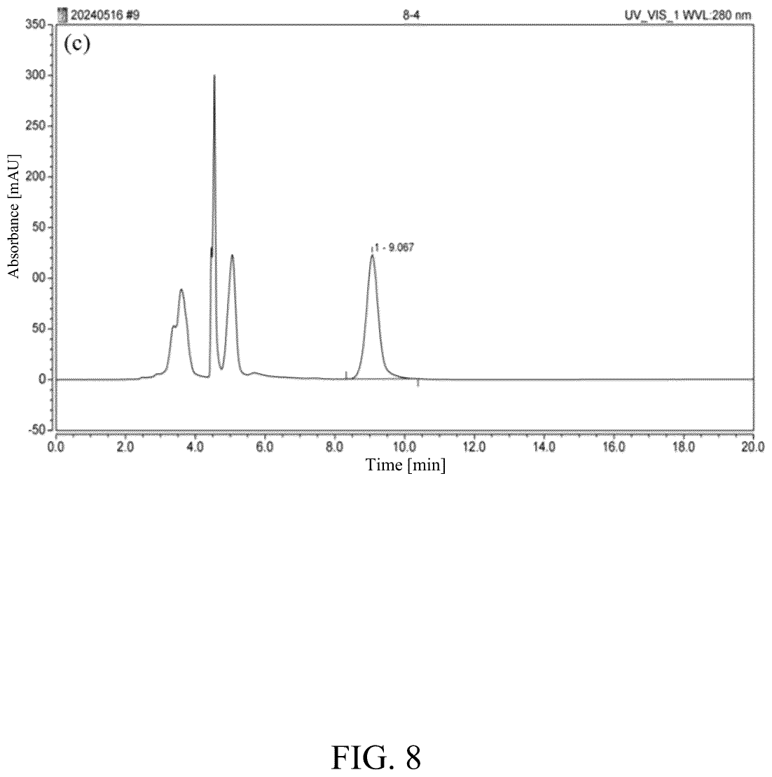

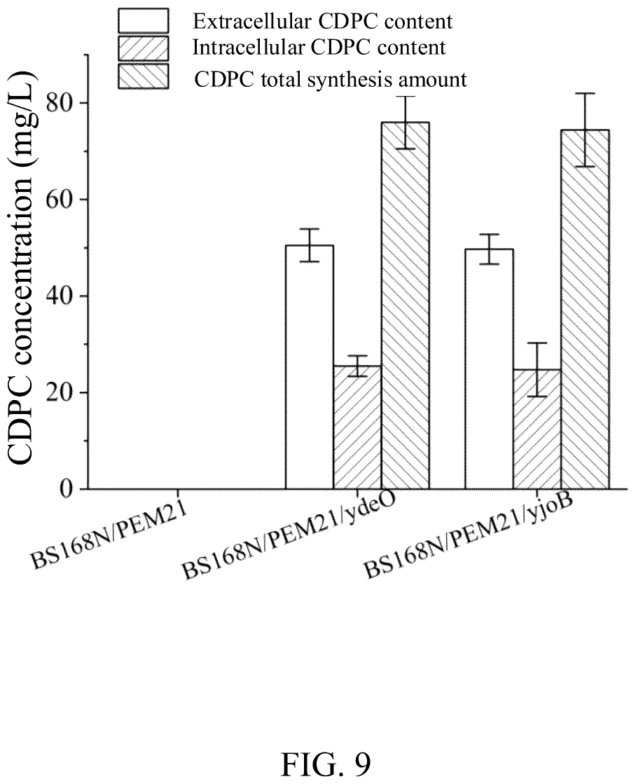

Wherein the reference labels shown in FIG. 1 are as below: Glucose: glucose; Glucose-6P: glucose-6-phosphate; Frucose-6P: fructose-6-phosphate; Frucose-1,6P₂: fructose-1,6-diphosphate; Glyceraldehyde-3P: glyceraldehyde 3-phosphate; Glycerone-P: dihydroxyacetone phosphate; Glycerol: glycerol; Glycerol-3P: glycerol-3-phosphate; 1-Acyl-sn-glycerol-3P: 1-acyl-sn-glycerol-3-phosphate; 1,2-Diacyl-sn-glycerol-3P: 1,2-diacyl-sn-glycerol-3-phosphate; CDP-diacylglycerol: CDP-diacylglycerol; Phosphatidyl-L-serine: phosphatidyl-L-serine; Phosphatidylethanolamine: phosphatidylethanolamine; Phosphatidyl-N-methylethanolamine: phosphatidyl-N-methylethanolamine; Phosphatidyl-N-dimethylethanolamine: phosphatidyl-N-dimethylethanolamine; Phosphatidylcholine: phosphatidylcholine; 1-Acyl-sn-glycero-3-phosphocholine: 1-Acyl-sn-glycero-3-phosphocholine; sn-Glycero-3-phosphocholine: sn-Glycero-3-phosphocholine; Choline: choline; Phosphocholine: choline phosphate; CDP-choline: cytidine diphosphate-choline;

FIG. 2 shows the growth curves of three recombinant strains BS168N/PEM21, BS168N/PEM21/ydeO, and BS168N/PEM21/yjoB in Embodiment 3 of the present disclosure;

FIG. 3 shows the high-performance liquid chromatography chromatogram of the supernatant of the fermentation broth of recombinant bacterium BS168N/PEM21 in Embodiment 3 of the present disclosure;

FIG. 4 shows the high-performance liquid chromatography chromatogram of the supernatant from the fermentation broth of recombinant strain BS168N/PEM21/ydeO in Embodiment 3 of the present disclosure;

FIG. 5 shows the high-performance liquid chromatography chromatogram of the supernatant from the fermentation broth of recombinant strain BS168N/PEM21/yjoB in Embodiment 3 of the present disclosure;

FIG. 6 shows the high-performance liquid chromatography chromatogram of the supernatant obtained from lysed cell pellets of recombinant bacterium BS168N/PEM21 in Embodiment 3 of the present disclosure;

FIG. 7 shows the high-performance liquid chromatography chromatogram of the supernatant obtained from lysed cell pellets of recombinant strain BS168N/PEM21/ydeO in Embodiment 3 of the present disclosure;

FIG. 8 shows the high-performance liquid chromatography chromatogram of the supernatant obtained from lysed cell pellets of recombinant strain BS168N/PEM21/yjoB in Embodiment 3 of the present disclosure;

FIG. 9 shows the extracellular and intracellular CDPC content, as well as the total synthesis of CDPC, of three recombinant strain BS168N/PEM21, BS168N/PEM21/ydeO, and BS168N/PEM21/yjoB in Embodiment 3 of the present disclosure.

### DETAILED DESCRIPTION OF THE EMBODIMENTS

In order to enable skilled person in the art to better understand the present disclosure, the present disclosure will be further elaborated in conjunction with specific embodiments.

### 1. Preparation and Sources of Strains, Plasmids, and Culture Media.

All strains and plasmid information involved in the present disclosure are detailed in Table 1, and the primers were synthesized by GenScript company.

LB medium: tryptone 10 g/L, yeast extract 5 g/L NaCl 10 g/L, add 15 g/L agar powder to solid culture medium, and add 2 μg/mL erythromycin (E2), 16 μg/mL neomycin (N16), and 8 μg/mL chloramphenicol (C8) according to actual need for screening Bacillus subtilis transformants and seed cultivation.

Fermentation medium: glucose 40 g/L, tryptone 10 g/L, yeast extract 5 g/L, NaCl 10 g/L, MgSO₄·7H₂O 1 g/L, CMP 0.02 g/L, xylose 10 g/L, and pH 6.0-8.0.

TABLE 1
Strains and plasmids involved in the experiment

| | | |
|---|---|---|
| Name | Genetic background | Source |

Strains

| | | |
|---|---|---|
| S. cerevisiae | Saccharomyces cerevisiae | Laboratory |
| | | preservation |
| BS168N | trpC 2, ΔaraR::Pₐᵣₐ-neo | Laboratory |
| | | preservation |
| BS168NCm | trpC 2, ΔaraR::neo, ΔyrpC::cat-araR | Laboratory |
| | | preservation |
| BSC1-2 | BS168N, ΔyrpC::TP2-CCT-CKI^{E216G} | Laboratory |
| | | preservation |
| BS168N/PEM21 | BS168N, ΔlacA::P_{xyl}-PEM2-PEM1 | Constructed |
| | | in this present |
| | | disclosure |
| BS168N/PEM21/ydeO | BS168N/PEM21, | Constructed |
| | ΔydeO::TP2-CCT-CKI^{E216G} | in this present |
| | | disclosure |
| BS168N/PEM21/yjoB | BS168N/PEM21, | Constructed |
| | ΔyjoB::TP2-CCT-CKI^{E216G} | in this present |
| | | disclosure |

Plasmid

| | | |
|---|---|---|
| pJMP1 | Bacillus subtilis dCas9 expression vector; | Laboratory |
| | integrates into lacA | preservation |

### 2. Reagents and Instruments.

High fidelity DNA polymerase 2×Phanta Flash Master Mix (Dye Plus) and high-purity heat-resistant DNA polymerase 2×Taq Master Mix (Dye Plus) were both purchased from Nanjing Vazyme Biotechnology Co., Ltd; the plasmid extraction kit, yeast genomic DNA extraction kit, and bacterial genomic DNA extraction kit were all purchased from Tiangen Biochemical Technology (Beijing) Co., Ltd; standard CDP-choline sodium was purchased from Sigma Aldrich (USA); the remaining biochemical reagents are domestic analytical grade reagents.

Instruments used: PCR instrument (LifeECO), microplate reader (Thermo), ultrasonic cell disruptor (Ningbo Xinzhi), high-performance liquid chromatography chromatogram (Agilent).

### 3. Primers.

The primers used for PCR are shown in Table 2.

TABLE 2
PCR Primer Sequences

| | Sequence | |
|---|---|---|
| Primers | name | Sequence (5′→3′) |
| lacA-U1 | SEQ ID | CAATAACGCTGGATACATCTG |
| | No. 1 | |
| lacA-U2 | SEQ ID | TGAATCTGCTGTCATCCAT |
| | No. 2 | |
| lacA-P1q | SEQ ID | ATGGATGACAGCAGATTCACAGTGTATTTAC |
| | No. 3 | TTGAGAGGAG |
| lacA-P2 | SEQ ID | CATTTCCCCCTTTGATTITTAG |
| | No. 4 | |
| PEM2-1q | SEQ ID | TCTAAAAATCAAAGGGGGAAATGAAGGAGT |
| | No. 5 | CAGTCCAAGAGAT |
| PEM2-2 | SEQ ID | AGAAATAGAGAACTTACATATTC |
| | No. 6 | |
| PEM1-1q | SEQ ID | GAATATGTAAGTTCTCTATTTCTAAAGGAGG |
| | No. 7 | AATAAAAAATGTCCAGITGTAAAACCACT |
| PEM1-2 | SEQ ID | TCAAGCAAGACTATCAAGC |
| | No. 8 | |
| lacA-ED1q | SEQ ID | CGCTTGATAGTCTTGCTTGAGAATTGAACCT |
| | No. 9 | CTACCACAA |
| lacA-ED2 | SEQ ID | GGGTGATGTCTAAGGTTGA |
| | No. 10 | |
| CX-PP1 | SEQ ID | TTGAGAGGAGACAGTAGACTT |
| | No. 11 | |
| CX-PP2 | SEQ ID | GCATCTAACTTGACCCAGA |
| | No. 12 | |
| CX-PP3 | SEQ ID | TCCTATGAGCAATGGCAAT |
| | No. 13 | |
| CX-PP4 | SEQ ID | ATTGCTAGTCTCGTTTCCTT |
| | No. 14 | |
| CX-PP5 | SEQ ID | CGTTTCCGCCATTCTTTG |
| | No. 15 | |
| ydeO-U1 | SEQ ID | CTTCATATAACGCAGCATCA |
| | No. 16 | |
| ydeO-U2q | SEQ ID | ACCATCAACGCAACCATAAACTGAACCAAC |
| | No. 17 | GACGCTAAGA |
| ydeO-PCC2 | SEQ ID | AGATGTTAGCGACAGTAATAG |
| | No. 18 | |
| ydeO-D1q | SEQ ID | TCTATTACTGTCGCTAACATCTCGGTGAAGG |
| | No. 19 | TCTGAATCA |
| ydeO-D2 | SEQ ID | AGGAAGGCTGATAGGTCAT |
| | No. 20 | |
| ydeO-CR1q | SEQ ID | CATGACCTATCAGCCTTCCTTCTTCAACTAA |
| | No. 21 | AGCACCCAT |
| ydeO-CR2 | SEQ ID | TTATTCATTCAGTTTTCGTG |
| | No. 22 | |
| ydeO-G1q | SEQ ID | GCACGAAAACTGAATGAATAACGGCTAACAT |
| | No. 23 | AGAAGGAGAT |
| ydeO-G2 | SEQ ID | GCTACATCATACACGGTAAC |
| | No. 24 | |
| CX-CC5 | SEQ ID | ATCTCTGGTGCGATGACA |
| | No. 25 | |
| yjoB-U1 | SEQ ID | GACGATAAACCCAAACACAA |
| | No. 26 | |
| yjoB-U2q | SEQ ID | ACCATCAACGCAACCATAAACCGCAACTCTT |
| | No. 27 | TCATTCTCTT |
| yjoB-PCC2 | SEQ ID | TGGAGTTAGCCACAGTAATAG |
| | No. 28 | |
| yjoB-D1q | SEQ ID | TCTATTACTGTGGCTAACTCCATCATTGGCAC |
| | No. 29 | GAAGAAGG |
| yjoB-D2 | SEQ ID | GATTCTGCGGACGGTGTA |
| | No. 30 | |
| yjoB-CR1q | SEQ ID | GTACACCGTCCGCAGAATCTCTTCAACTAAA |
| | No. 31 | GCACCCAT |
| yjoB-G1q | SEQ ID | GCACGAAAACTGAATGAATAAGGTTGATTG |
| | No. 32 | GAGCCTGAG |
| yjoB-G2 | SEQ ID | CGTCCTGCGTATTGAGAA |
| | No. 33 | |
| 2-yjoB-U1 | SEQ ID | TCTTCCTGATGCTGGTGAA |
| | No. 34 | |

The present disclosure provides a recombinant engineered strain, preparation method, and application for de novo synthesis of CDP-choline using glucose as a substrate, as follows:

In the present disclosure, first the phosphatidylethanolamine N-methyltransferase gene PEM1 and phosphatidylethanolamine/phosphatidyl-N-methylethanolamine N-methyltransferase gene PEM2 of S. cerevisiae were integrated with the xylose induced promoter P_{xyl} to form an artificial operon P_{xyl}-PEM2-PEM1, which was then integrated into the genome of Bacillus subtilis 168N, aiming to open up the synthesis pathway from glucose to choline. Then, the artificial operon TP2-CCT-CKI constructed in patent CN116790466B was integrated into the genome of Bacillus subtilis expressing P_{xyl}-PEM2-PEM1, opening up the synthetic pathway from choline to CDPC, and then shake flask fermentation culture was performed on the constructed recombinant engineered strain, measuring the growth of the strain and the synthesis of CDPC separately.

### Embodiment 1

Construction of recombinant strain BS168N/PEM21: co-expressing the PEM2 and PEM1 genes of S. cerevisiae at the lacA site on the BS168N genome.

Firstly, taking the genome of BS168N as a template, primers lacA-U1/lacA-U2 were used to amplify the fragment U containing the upstream homologous arm (as shown in SEQ ID No. 35, 1309 bp).

Taking the plasmid pJMP1 as a template, primers lacA-P1q/lacA-P2 were used to amplify the fragment P containing xylose inducible promoter P_{xyl} (as shown in SEQ ID No. 36, 426 bp).

Primers lacA-ED1q/lacA-ED2 were used to amplify the fragment ED containing the erythromycin resistance gene and downstream homologous arm (as shown in SEQ ID No. 37, 1976 bp).

Taking the genome of S. cerevisiae as a template, primers PEM2-1q/PEM2-2 and PEM1-1q/PEM1-2 were used to amplify the fragment M2 containing the PEM2 gene sequence (as shown in SEQ ID No. 38, 631 bp) and the fragment M1 containing the PEM1 gene sequence (as shown in SEQ ID No. 39, 2626 bp), respectively.

Secondly, primers lacA-P1q/PEM1-2 were used to splice the fragment P, the fragment M2, and the fragment M1 together to form the fragment PM2M1 (3683 bp) though overlap-PCR method; then, primers lacA-U1/lacA-ED2 were used to splice the fragment U, the fragment PM2M1, and the fragment ED into the fragment UPM2M1ED (as shown in SEQ ID No.40, 6968 bp).

Finally, the fragment UPM2M1ED was transformed into competent cells of the receptor bacterium BS168N and coated onto LBE2 solid plates. The grown transformants were selected and transferred to LBE2 liquid medium for shaking culture for 2 hours. Primers CX-PP1/CX-PP5 were used for bacterial liquid PCR verification. The correct transformant was one with a band size of 3814 bp.

A correct transformant is randomly selected and transferred into a test tube containing 5 mL of LBE2 liquid medium for overnight culture. Extracting its genome as a template, and primers CX-PP1/CX-PP5 were used for amplification. Then, the PCR product was sent for testing, with sequencing primers as follows: CX-PP1/CX-PP2/CX-PP3/CX-PP4/CX-PP5.

CX-PP1/CX-PP2/CX-PP3/CX-PP4/CX-PP5 were sequenced, and a recombinant strain without additional point mutations was obtained. The strain was named BS168N/PEM21 and deposited.

### Embodiment 2

Construction of recombinant engineered strain BS168N/PEM21/ydeO and BS168N/PEM21/yjoB: co-expressing The CCT and CKI genes of S. cerevisiae at ydeO and yjoB sites on the BS168N/PEM2 genome, respectively.

### (1) Construction of Recombinant Strain BS168N/PEM21/ydeO

Firstly, taking the genome of BS168NCm as a template, primers ydeO-U1/ydeO-U2q, ydeO-D1q/ydeO-D2, ydeO-CR1q/ydeO-CR2, and ydeO-D1q/ydeO-G2 were used to amplify the fragment U containing upstream homologous arms (as shown in SEQ ID No. 41, 1302 bp), the fragment D containing homologous recombination (as shown in SEQ ID No. 42, 1005 bp), the fragment CR containing chloramphenicol resistance gene and arabinose operon repressor gene (as shown in SEQ ID No. 43, 2069 bp), and the fragment G containing downstream homologous arms (as shown in SEQ ID No. 44, 514 bp).

Taking the genome of BSC1-22 as a template, primers TP2-1/ydeO-PCC2 were used to amplify the fragment PCC (as shown in SEQ ID No. 45, 3329 bp) containing the constitutive strong promoter TP2, CCT gene and CKI gene derived from S. cerevisiae.

Secondly, primers ydeO-U1/ydeO-PCC2 were used to splice the fragment U, the fragment PCC, and the fragment D into the fragment UPCCD (5636 bp) through the overlap-PCR method; then, primers ydeO-U1/ydeO-G2 were used to splice the fragment UPCCD, the fragment CR, and the fragment G into the fragment UPCCDCRG (as shown in SEQ ID No. 46, 8219 bp).

Finally, the fragment UPCCDCRG was transformed into competent cells of the receptor bacterium BS168N/PEM21 and coated onto LBC8 solid plates. The grown transformants were selected and transferred to LBC8 liquid medium for shaking culture for 2 hours. Primers CX-CC3/ydeO-D2 were used for bacterial liquid PCR verification. The correct transformant was the one with a band size of 2395 bp.

A correct transformant was randomly selected and transferred to the antibiotic-free LB liquid medium for shaking cultivation for 4 hours, and then 1 μL of bacterial solution was taken to coat onto a LBN16 solid plate. The grown transformants were selected and transfer to the LBN16 liquid medium for shaking cultivation for 2 hours, followed by another round of bacterial liquid PCR validation. Finally, the two rounds of validated single colonies were selected to incubate into in a test tube containing 5 mL of LBN16 liquid medium for overnight culture, extracting its genome as a template, and primers TP2-1/ydeO-PCC2 were used for amplification, with a band size of 3329 bp. Sending the PCR product for testing, and the primers were TP2-1/CCT-2/CX-CC3/CX-CC5/ydeO-PCC2 for sequencing. As a result, a recombinant strain with the same point mutation as the CKI gene mutation of strain BSC1-22 in patent CN202310882430. X was obtained. The strain was named BS168N/PEM21/ydeO and deposited, and the sequence of primers TP2-1/CCT-2/CX-CC3 can be found in patent CN116790466B.

### (2) Construction of Recombinant Strain BS168N/PEM21/yjoB

Firstly, taking the genome of BS168NCm as the template, yjoB-U1/yjoB-U2q, yjoB-D1q/yjoB-D2, yjoB-CR1q/ydeO-CR2, yjoB D1q/yjoB-G2 were used to amplify the fragment U (as shown in SEQ ID No. 47, 1362 bp), the fragment D (as shown in SEQ ID No. 48, 808 bp), the fragment CR (as shown in SEQ ID No. 43, 2069 bp), and the fragment G (as shown in SEQ ID: 49, 697 bp); taking the genome of BSC1-22 as the template, TP2-1/yjoB-PCC2 was used to amplify the fragment PCC (as shown in SEQ ID No. 50, 3329 bp).

Secondly, primers yjoB-U1/yjoB-PCC2 were used to splice the fragment U, the fragment PCC, and the fragment D into the fragment UPCCD (5333 bp) through the overlap-PCR method; then, primers 2-yjoB-U1/yjoB-G2 were used to splice the fragment UPCCD, the fragment CR, and the fragment G into the fragment UPCCDCRG (as shown in SEQ ID No. 51, 8099 bp).

Finally, the fragment UPCCDCRG was transformed into competent cells of the receptor bacterium BS168N/PEM21 and coated onto LBC8 solid plates. After two rounds of screening, the correct transformant was obtained, extracting its genome as a template, and primers TP2-1/yjoB-PCC2 were used for amplification, with a band size of 3329 bp. Sending the PCR product for testing, and the sequencing primers were TP2-1/CCT-2/CX-CC3/CX-CC5/yjoB-PCC2. As a result, a recombinant strain with the same point mutation as the CKI gene mutation of strain BSC1-22 in patent CN202310882430. X was obtained, and the strain was named BS168N/PEM21/yjoB and deposited.

### Embodiment 3

Two artificial operons, P_{xyl}-PEM2-PEM1 and TP2-CCT-CKI, were integrated into the genome of Bacillus subtilis for expression and detect the synthesis of CDPC.

### (1) Shake Flask Fermentation Culture and Biomass Determination

Newly activated single colonies of BS168N/PEM21, BS168N/PEM21/ydeO, and BS168N/PEM21/yjoB were selected from the solid plate of LBN16+E2 and transferred to a test tube containing 5 mL of LBN16+E2 liquid medium, shaking at 37° C. and 200 r/min for 12 hours. The culture was transferred to a 250 mL conical flask containing 30 mL of fermentation medium at 1% inoculated volume, then conducting shaking culture at 37° C. and 220 r/min for 26 hours. After 3 hours of fermentation, xylose with a final concentration of 10 g/L was added for induction.

1 mL of fermentation broths that has been fermented for 6 hours, 11 hours, and 26 hours were taken respectively, then performing centrifuge to remove the supernatant, the cell pellet was resuspended and diluted appropriately after washed with deionized water to measure the OD₆₀₀ value of the bacterial suspension.

The growth curves of three recombinant strains BS168N/PEM21, BS168N/PEM21/ydeO, and BS168N/PEM21/yjoB are shown in FIG. 2.

### (2) Extraction and Detection of CDPC

30 mL of 26 hour fermentation broth was taken, conducting centrifuge at 8000 rpm and 4° C. for 10 minutes, and 1 mL of supernatant was taken for measuring the extracellular CDPC content. The remaining cell pellet was washed once with phosphate buffer solution at pH 7.4, then added to the cell pellet into 5 mL of phosphate buffer solution to resuspend, and then cell lysis was conducted by an ultrasonic cell disruptor, performing centrifuge at 8000 rpm and 4° C. for 10 minutes, and 1 mL of supernatant was taken to determine the intracellular CDPC content.

The HPLC detection conditions are detailed in patent CN116790466B.

FIGS. 3-5 show the high-performance liquid chromatography chromatograms of the fermentation supernatant of recombinant strain BS168N/PEM21, BS168N/PEM21/ydeO, and BS168N/PEM21/yjoB, respectively.

FIGS. 6-8 show the high-performance liquid chromatography chromatograms (all concentrated 6-fold) of the supernatant obtained from lysed cell pellets of recombinant strain BS168N/PEM21, BS168N/PEM21/ydeO, and BS168N/PEM21/yjoB, respectively.

### (3) the Effect of Integrating PEM2-PEM1 and CCT-CKI Expression on CDPC Synthesis

B. Subtilis lacks a synthetic pathway from PE to PC and cannot synthesize choline from scratch, and lacks a synthetic pathway from choline to CDPC. While there is a complete CDPC pathway in S. cerevisiae. Therefore, in this embodiment, BS168N was used as the starting strain, and firstly, integrate the phosphatidylethanolamine N-methyltransferase gene PEM1 and the phosphatidylethanolamine/phosphatidyl-N-methylethanolamine N-methyltransferase gene PEM2 of S. cerevisiae with the xylose induced promoter P_{xyl} to form an artificial operon P_{xyl}-PEM2-PEM1, which was integrated into the genome of Bacillus subtilis 168N to obtain the recombinant strain BS168N/PEM21, aiming to open up the synthesis pathway from glucose to choline. Subsequently, the choline kinase gene CKI, the phosphocholine cytidylyltransferase gene CCT were combined with the constitutive strong promoter TP2 to form an artificial operon TP2-CCT-CKI, which was integrated into the ydeO and yjoB sites of the BS168N/PEM21 genome, respectively, to obtain recombinant strain BS168N/PEM21/ydeO and BS168N/PEM21/yjoB, aiming to open up the synthetic pathway from choline to CDPC. Due to the simultaneous integrant expression of four genes, namely PEM1, PEM2, CKI, and CCT in the BS168N/PEM21/ydeO and BS168N/PEM21/yjoB strains, thus, the synthetic pathway from glucose to CDPC has theoretically been opened up.

BS168N/PEM21, BS168N/PEM21/ydeO, and BS168N/PEM21/yjoB were subjected to shake flask fermentation culture. The growth results of the strains are shown in Table 3. The total synthesis of extracellular, intracellular, and CDPC of different recombinant strains after 26 hours of fermentation is shown in Table 4 and FIG. 9.

TABLE 3
Growth of strains

| Fermentation | OD₆₀₀ | OD₆₀₀ | OD₆₀₀ |
|---|---|---|---|
| time (h) | (BS168N/PEM21) | (BS168N/PEM21/ydeO) | (BS168N/PEM21/yjoB) |
| 6 | 4.11 ± 0.66 | 3.43 ± 0.19 | 3.38 ± 0.51 |
| 11 | 7.69 ± 0.07 | 7.63 ± 0.03 | 6.82 ± 0.19 |
| 26 | 9.51 ± 0.31 | 7.24* ± 0.27 | 7.27* ± 0.30 |

Note:
*indicates there is a significant difference (P < 0.05) compared to BS168N/PEM21.

TABLE 4
Total synthesis of extracellular, intracellular, and CDPC of
different recombinant strains after 26 hours of fermentation

| | Extracellular CDPC | Intracellular CDPC | CDPC total synthesis |
|---|---|---|---|
| Strains | production (mg/L) | production (mg/L) | amount (mg/L) |
| BS168N/PEM21 | — | — | — |
| BS168N/PEM21/ydeO | 50.5^{#} ± 3.4 | 25.5^{#} ± 2.1 | 76.0^{#} ± 5.5 |
| BS168N/PEM21/yjoB | 49.7^{#} ± 3.1 | 24.7^{#} ± 4.5 | 74.4^{#} ± 7.6 |

Note:
^{#}indicates a highly significant difference (P < 0.01) compared to BS168N/PEM21.

The results in Table 3 show that during the period of 12-26 hours, the growth of strains BS168N/PEM21/ydeO and BS168N/PEM21/yjoB decreased compared to BS168N/PEM21.

In addition, the total synthesis of extracellular, intracellular, and CDPC by different recombinant strains in Table 4 showed that after 26 hours of fermentation, no CDPC was detected in the supernatant and cell pellet of BS168N/PEM21; CDPC was detected in the supernatant and cell pellet of BS168N/PEM21/ydeO and BS168N/PEM21/yjoB.

Moreover, the extracellular CDPC content of recombinant strain BS168N/PEM21/ydeO was 50.5±3.4 mg/L, the intracellular CDPC content was 25.5±2.1 mg/L, and the total CDPC synthesis was 76.0±5.5 mg/L. The extracellular CDPC content of recombinant strain BS168N/PEM21/yjoB was 49.7±3.1 mg/L, the intracellular CDPC content was 24.7±4.5 mg/L, and the total CDPC synthesis was 74.4±7.6 mg/L.

The above results indicate that introducing PEM2-PEM1 and CCT-CKI into the genome of B. subtilis for expression indeed opens up the CDPC synthesis pathway starting from glucose, and approximately 66% of CDPC is secreted extracellular; the expression of CCT-CKI is not related to the gene locus (ydeO or yjoB) integrated and inserted, resulting in little difference in yield between the two recombinant strains; and the growth of the last two strains decreased after 26 hours, this is because some glucose is used to synthesize CDPC, resulting in a decrease in glucose used for growth.

The present disclosure is the first to utilize the engineered B. Subtilis uses glucose as a substrate and can synthesize CDPC from scratch without the need to add precursors choline chloride or choline phosphate, laying a research foundation for reducing the production cost of CDPC in the future.

Sequence list

SEQ ID No. 35
Caataacgctggatacatctgcccctttgataaaatccttgttcatcccctctacttttttaacaaaaagatcctctgccc
gaagctctgacacgtgcttctccttctcaatggcagcggcatatcctgttgaactacatgcactccacacgatggcagccgc
aaagaacattttcactttgcttttcatgatttcatccccccaagacttagcaagcgttttcattctatagaaaaaagaatccgccc
atatcgagcggagcatcagcctaatgtgtgtttacgacaattctcacttcatacttttccatcgtcaggtcgcctgacaatatgtc
tcctgtcattatgtccttcacactctgatcaaacgtgaccagctgtttttcttccgtgaaattcatgacaaaaatataatcattgtcc
tgatcctgcctcgcttgtacggagacgccttttccgtgccgaaccggaaaaactggagagagagacaggtctgtgatcaga
ccctcatagaaatcacgctgaaattgatcctccaaacgcgcgccgataaaatacgccttgccctgctgatactcatggcttgt
gaccgctggcgtgcgcgcataaaaatcttcttgatacaccgcttccactgaagctgtctttacatcaatcacggttgcataatc
cttcatttcatatatttggctgcggtagctgacagcgtttcgatccttcggatacagggtgtccgtttcaagaggctcaactcca
aatatagcttgaagatccggatgccatccgcctgtgtatgttaagtcatgctcattcacaaccccgctgatatacgtcatgact
aaggtgccgccgtcagccgtaaacgcttttaaacgggaaacggtgtcctcgctgattaaatacagcatcgggacgatcagc
agtttatatggtgaaaagtcttgttctttcgtgatgacgtcgacagggatatcgtgttcccagaatgtgcggtaatgctgctgaa
gcgtttgcggataacgttttgtcgccttcgcaaacccctgagcatcctcgagcgcccaatgattttcccagtcatataaaatcg
cggtttgagccggcctcttcgttccgacaacttcggacagccgttccaatgtctcgcctaccttggccacttcttgaaagacg
cggttcttcgggctattgtcatgatccacaaccgctccgtgtaatttttctgatgacccccgtgatttgcggtattggaaataga
gaacgctgtccgagccgtgggcaatcatttgcatggatgacagcagattca.
SEQ ID No. 36
Cagtgtatttacttgagaggagacagtagacttgtttaatcctgtaatctcagagagagttgccctggagacagggg
agttcttcaaaatttcatctaatattaatttttgattcattttttttactaaagcttgatctgcaatttgaataataaccactcctttgttt
atccaccgaactaagttggtgttttttgaagcttgaattagatatttaaaagtatcatatctaatattataactaaattttctaaaaaaa
acattgaaataaacatttattttgtatatgatgagataaagttagtttattggataaacaaactaactcaattaagatagttgatgg
ataaacttgttcacttaaatcaaagggggaaatgacaaatggtccaaactagtgatatctaaaaatcaaagggggaaatg.
SEQ ID No. 37
Gaattgaacctctaccacaaaagaaaaacgaaatgatacaccaatcagtgcaaaaaaagatataatgggagataa
gacggttcgtgttcgtgctgacttgcaccatatcataaaaatcgaaacagcaaagaatggcggaaacgtaaaagaagttatg
gaaataagacttagaagcaaacttaagagtgtgttgatagtgcagtatcttaaaattttgtataataggaattgaagttaaattag
atgctaaaaatttgtaattaagaaggagtgattacatgaacaaaaatataaaatattctcaaaactttttaacgagtgaaaaagt
actcaaccaaataataaaacaattgaatttaaaagaaaccgataccgtttacgaaattggaacaggtaaagggcatttaacga
cgaaactggctaaaataagtaaacaggtaacgtctattgaattagacagtcatctattcaacttatcgtcagaaaaattaaaact
gaatactcgtgtcactttaattcaccaagatattctacagtttcaattccctaacaaacagaggtataaaattgttgggagtattc
cttaccatttaagcacacaaattattaaaaaagtggtttttgaaagccatgcgtctgacatctatctgattgttgaagaaggattc
tacaagcgtaccttggatattcaccgaacactagggttgctcttgcacactcaagtctcgattcagcaattgcttaagctgcca
gcggaatgctttcatcctaaaccaaaagtaaacagtgtcttaataaaacttacccgccataccacagatgttccagataaatat
tggaagctatatacgtactttgtttcaaaatgggtcaatcgagaatatcgtcaactgtttactaaaaatcagtttcatcaagcaat
gaaacacgccaaagtaaacaatttaagtaccgttacttatgagcaagtattgtctatttttaatagttatctattatttaacgggag
gaaataattctatgagtcgcttttgtaaatttggaaagttacacgttactaaagggaatgtagataaattattaggtatactactga
cagcttccaaggagctaaagaggtccctagactctagacccggggatctctgcagtgagatctggtaatgactctctagctt
gaggcatcaaataaaacgaaaggctcagtcgaaagactgggcctttcgttttatctgttgtttgtcggtgaacgctctcctgag
taggacaaatccgccgctctagctaagcagaaggccatcctgacggatggcctttttgcgtttctacaaactcttgttaactct
agagctgcctgccgcgtttcggtgatgaagatcttcccgatgattaattaattcagaacgctcggttgccgccgggcgtttttt
atgcagcaatggcaagaacgttgctctagagcggccgcatcgattcacagtggcaatctcccccgtattcgtttgaaatgtg
ccacattaacagcgccgggtgatgtccgtatcgttctgctaataagcggttgatgtgccgtgttttttctcggtagactttagat
gtgaggcagtggttgtgccttccgccgtgcagctgtttgacgcgggaggcattgacgcgcaaaacttccggataggtttgc
gacagccaggccggacgggctccgctcggcgttgctaatatgacccggccgcctatactgtgaatccgctcaaaaatatc
atccagccattcaaattgatatacgccctcctccggctcaagtgcgctccatgcaaaaatgccgacagaaaacgtattcgtat
gagaaagcttcatcagtttgatatcgtcagctaaaatatcgggccgatccagccactgatcggggttgtagtctcccccatgg
agcataaattttgcttttgttacgtgcgttttttcaaccttagacatcaccc.
SEQ ID No. 38
Aaggagtcagtccaagagatcatccagcaactcatccacagtgtcgatttacagtcttccaagttccagctggccatt
gtgtgcacgatgttcaatcctatcttttggaacatcgttgcaagaatggaataccacaagcattctctcaccaagatgtgtggt
ggggccagaaagggctgttacatgttggcggcgaccatattttcgctaggtatcgtcagagacatggtgtacgagtctgcat
tgcgtgaacagcctacgtgttctctgatcacgggcgagaactggaccaagctgggtgtggctctctttggtttggggcaagt
gcttgttttgagttccatgtacaagctgggtatcacagggacgtacttgggtgactatttcggcatcctgatggatgagagagt
caccggcttccccttcaacgtttccaacaaccccatgtaccagggttccactttgtccttcttgggcatagccctttacaaagg
aaagcctgcggggctggttgtttctgccgtagtttacttcatgtacaagatcgctcttcgttgggaagaaccttttactgccatg
atctacgctaaccgtgataaggccaaaaagaatatgtaagttctctatttct.
SEQ ID No. 39
aaaggaggaataaaaaatgtccagttgtaaaaccactttgtctgaaatggttggttctgtgacaaaagataggggcac
tatcaacgtcgaagccagaacacgttcgagtaatgtaactttcaaaccacctgtaacccacgatatggtgcgttcgcttttcga
tccaactttgaagaaatctctgctggaaaaatgcatcgcgctagctattatatcgaattttttcatttgttattgggtcttccaaag
gtttggcttacagtttaccaaatacttttttctggtacagtatttattttggagaattgcttataatttaggtattggactggttctgcat
taccagtcacattatgaaacactaacgaattgtgctaaaactcatgcgattttcagcaaaataccacagaataaagatgctaat
tcgaatttctcaacaaattccaattccttttcagaaaaattttggaattttattaggaagttctgtcaatatgagattaggtctaaaat
gccaaaggagtatgatttatttgcctatccagaagagatcaacgtctggttgatatttcgccagtttgtcgatttaatcttgatgc
aagattttgtgacttacattatttacgtttacctttctattccatatagctgggttcaaatcttcaactggagatctttgctaggtgtta
ttttgattttatttaacatctgggtcaagttagatgcgcatcgcgtggttaaagattatgcttggtactggggtgatttcttctttttg
gaagaatctgaattgatttttgatggtgtcttcaacatctctccacatccaatgtattctattggttatttgggttattatggcctatc
attgatttgtaatgactacaaggttcttctggtgtccgtatttggacattattctcaatttttgtttctgaaatacgtcgaaaatcctc
atattgaaagaacgtatggtgatggcactgattccgattctcaaatgaatagcagaattgatgacttgatatcaaaagagaatt
acgattattcaagacctctaattaacatgggtctttcttttaacaacttcaataagctaagatttactgactatttcacaattggtac
cgtagcggcacttatgttaggaacaatcatgaatgctaggtttatcaatttaaattacctctttattaccgtttttgtaacaaaactg
gtttcatggttatttatctcgacaatattgtacaagcaatctcagtctaaatggttcacgagattgtttttggagaatggttacaca
caagtttattcctatgagcaatggcaatttatttacaattattatttggtattaacctacacacttatgataattcatacagggcttca
aatttggagtaatttttccaacataaacaacagtcaattaatttttggtttaattcttgtggctttacaaacatggtgtgataaggaa
acgagactagcaatctctgattttggttggttttatggtgattttttcttgagcaactatatctcaactagaaagctgacttcccaa
ggtatctacagatatttgaatcacccagaagcggtattaggcgttgttggagtttggggtactgtattgatgacgaattttgccg
ttacaaatattatcttggctgttttatggacattgacaaattttattcttgtcaagtttattgaaacacctcatgttaataagatatatg
gtaaaactaaacgagttagtggtgttggaaaaactttactaggcttgaaacctctaagacaagtttcagatattgtcaatagaat
tgaaaatattataatcaaatcattagttgatgaaagcaagaactccaatggtggggcagaacttttgcccaaaaattatcaaga
taacaaagaatggaatattttaattcaggaagcaatggatagcgttgcaacaagattgagtccatactgtgaattgaaaattga
aaacgagcaggtagaaaccaattttgtcttgcctactccagtaactttgaattggaaaatgcccattgaactttataacggaga
tgattggattggtttgtataaagtcattgatacaagggcagatcgtgaaaaaacaagagttggatcaggtggtcattggagtg
cgacttctaaagattcatatatgaaccatggattaagacataaagaatctgtaacagaaataaaggcgactgaaaagtacgtt
caaggtaaagtgacattcgacacgtctttgctttattttgaaaatggtatctacgaatttaggtatcattctggtaactcccataag
gtgttattgatttctacaccatttgaaatttcattgccggtactaaataccacgacacctgaactgtttgagaaggatttgactga
gtttttgacgaaagtcaacgttttgaaggatggtaaatttcgtccattaggaaataagttcttcgggatggacagcttgaaacaa
ttgattaagaactcaattggcgtcgaactttcgtctgagtatatgaggagagtcaatggtgacgctcacgttatttcgcatcgc
gcttgggatataaaacaaacgcttgatagtcttgcttga.
SEQ ID No. 40
caataacgctggatacatctgcccctttgataaaatccttgttcatcccctctacttttttaacaaaaagatcctctgcccg
aagctctgacacgtgcttctccttctcaatggcagcggcatatcctgttgaactacatgcactccacacgatggcagccgca
aagaacattttcactttgcttttcatgatttcatccccccaagacttagcaagcgttttcattctatagaaaaaagaatccgcccat
atcgagcggagcatcagcctaatgtgtgtttacgacaattctcacttcatacttttccatcgtcaggtcgcctgacaatatgtctc
ctgtcattatgtccttcacactctgatcaaacgtgaccagctgtttttcttccgtgaaattcatgacaaaaatataatcattgtcctg
atcctgcctcgcttgtacggagacgccttttccgtgccgaaccggaaaaactggagagagagacaggtctgtgatcagacc
ctcatagaaatcacgctgaaattgatcctccaaacgcgcgccgataaaatacgccttgccctgctgatactcatggcttgtga
ccgctggcgtgcgcgcataaaaatcttcttgatacaccgcttccactgaagctgtctttacatcaatcacggttgcataatcctt
catttcatatatttggctgcggtagctgacagcgtttcgatccttcggatacagggtgtccgtttcaagaggctcaactccaaat
atagcttgaagatccggatgccatccgcctgtgtatgttaagtcatgctcattcacaaccccgctgatatacgtcatgactaag
gtgccgccgtcagccgtaaacgcttttaaacgggaaacggtgtcctcgctgattaaatacagcatcgggacgatcagcagt
ttatatggtgaaaagtcttgttctttcgtgatgacgtcgacagggatatcgtgttcccagaatgtgcggtaatgctgctgaagc
gtttgcggataacgttttgtcgccttcgcaaacccctgagcatcctcgagcgcccaatgattttcccagtcatataaaatcgcg
gtttgagccggcctcttcgttccgacaacttcggacagccgttccaatgtctcgcctaccttggccacttcttgaaagacgcg
gttcttcgggctattgtcatgatccacaaccgctccgtgtaatttttctgatgacccccgtgatttgcggtattggaaatagaga
acgctgtccgagccgtgggcaatcatttgcatggatgacagcagattcacagtgtatttacttgagaggagacagtagactt
gtttaatcctgtaatctcagagagagttgccctggagacaggggagttcttcaaaatttcatctaatattaatttttgattcatttttt
ttactaaagcttgatctgcaatttgaataataaccactcctttgtttatccaccgaactaagttggtgttttttgaagcttgaattag
atatttaaaagtatcatatctaatattataactaaattttctaaaaaaaacattgaaataaacatttattttgtatatgatgagataaa
gttagtttattggataaacaaactaactcaattaagatagttgatggataaacttgttcacttaaatcaaagggggaaatgacaa
atggtccaaactagtgatatctaaaaatcaaagggggaaatgaaggagtcagtccaagagatcatccagcaactcatccac
agtgtcgatttacagtcttccaagttccagctggccattgtgtgcacgatgttcaatcctatcttttggaacatcgttgcaagaat
ggaataccacaagcattctctcaccaagatgtgtggtggggccagaaagggctgttacatgttggcggcgaccatattttcg
ctaggtatcgtcagagacatggtgtacgagtctgcattgcgtgaacagcctacgtgttctctgatcacgggcgagaactgga
ccaagctgggtgtggctctctttggtttggggcaagtgcttgttttgagttccatgtacaagctgggtatcacagggacgtactt
gggtgactatttcggcatcctgatggatgagagagtcaccggcttccccttcaacgtttccaacaaccccatgtaccagggtt
ccactttgtccttcttgggcatagccctttacaaaggaaagcctgcggggctggttgtttctgccgtagtttacttcatgtacaa
gatcgctcttcgttgggaagaaccttttactgccatgatctacgctaaccgtgataaggccaaaaagaatatgtaagttctctat
ttctaaaggaggaataaaaaatgtccagttgtaaaaccactttgtctgaaatggttggttctgtgacaaaagataggggcacta
tcaacgtcgaagccagaacacgttcgagtaatgtaactttcaaaccacctgtaacccacgatatggtgcgttcgcttttcgatc
caactttgaagaaatctctgctggaaaaatgcatcgcgctagctattatatcgaattttttcatttgttattgggtcttccaaaggtt
tggcttacagtttaccaaatacttttttctggtacagtatttattttggagaattgcttataatttaggtattggactggttctgcatta
ccagtcacattatgaaacactaacgaattgtgctaaaactcatgcgattttcagcaaaataccacagaataaagatgctaattc
gaatttctcaacaaattccaattccttttcagaaaaattttggaattttattaggaagttctgtcaatatgagattaggtctaaaatg
ccaaaggagtatgatttatttgcctatccagaagagatcaacgtctggttgatatttcgccagtttgtcgatttaatcttgatgca
agattttgtgacttacattatttacgtttacctttctattccatatagctgggttcaaatcttcaactggagatctttgctaggtgttatt
ttgattttatttaacatctgggtcaagttagatgcgcatcgcgtggttaaagattatgcttggtactggggtgatttcttctttttgg
aagaatctgaattgatttttgatggtgtcttcaacatctctccacatccaatgtattctattggttatttgggttattatggcctatcat
tgatttgtaatgactacaaggttcttctggtgtccgtatttggacattattctcaatttttgtttctgaaatacgtcgaaaatcctcat
attgaaagaacgtatggtgatggcactgattccgattctcaaatgaatagcagaattgatgacttgatatcaaaagagaattac
gattattcaagacctctaattaacatgggtctttcttttaacaacttcaataagctaagatttactgactatttcacaattggtaccg
tagcggcacttatgttaggaacaatcatgaatgctaggtttatcaatttaaattacctctttattaccgtttttgtaacaaaactggt
ttcatggttatttatctcgacaatattgtacaagcaatctcagtctaaatggttcacgagattgtttttggagaatggttacacaca
agtttattcctatgagcaatggcaatttatttacaattattatttggtattaacctacacacttatgataattcatacagggcttcaaa
tttggagtaatttttccaacataaacaacagtcaattaatttttggtttaattcttgtggctttacaaacatggtgtgataaggaaac
gagactagcaatctctgattttggttggttttatggtgattttttcttgagcaactatatctcaactagaaagctgacttcccaaggt
atctacagatatttgaatcacccagaagcggtattaggcgttgttggagtttggggtactgtattgatgacgaattttgccgtta
caaatattatcttggctgttttatggacattgacaaattttattcttgtcaagtttattgaaacacctcatgttaataagatatatggta
aaactaaacgagttagtggtgttggaaaaactttactaggcttgaaacctctaagacaagtttcagatattgtcaatagaattga
aaatattataatcaaatcattagttgatgaaagcaagaactccaatggtggggcagaacttttgcccaaaaattatcaagataa
caaagaatggaatattttaattcaggaagcaatggatagcgttgcaacaagattgagtccatactgtgaattgaaaattgaaa
acgagcaggtagaaaccaattttgtcttgcctactccagtaactttgaattggaaaatgcccattgaactttataacggagatg
attggattggtttgtataaagtcattgatacaagggcagatcgtgaaaaaacaagagttggatcaggtggtcattggagtgcg
acttctaaagattcatatatgaaccatggattaagacataaagaatctgtaacagaaataaaggcgactgaaaagtacgttca
aggtaaagtgacattcgacacgtctttgctttattttgaaaatggtatctacgaatttaggtatcattctggtaactcccataaggt
gttattgatttctacaccatttgaaatttcattgccggtactaaataccacgacacctgaactgtttgagaaggatttgactgagtt
tttgacgaaagtcaacgttttgaaggatggtaaatttcgtccattaggaaataagttcttcgggatggacagcttgaaacaattg
attaagaactcaattggcgtcgaactttcgtctgagtatatgaggagagtcaatggtgacgctcacgttatttcgcatcgcgctt
gggatataaaacaaacgcttgatagtcttgcttgagaattgaacctctaccacaaaagaaaaacgaaatgatacaccaatca
gtgcaaaaaaagatataatgggagataagacggttcgtgttcgtgctgacttgcaccatatcataaaaatcgaaacagcaaa
gaatggcggaaacgtaaaagaagttatggaaataagacttagaagcaaacttaagagtgtgttgatagtgcagtatcttaaa
attttgtataataggaattgaagttaaattagatgctaaaaatttgtaattaagaaggagtgattacatgaacaaaaatataaaat
attctcaaaactttttaacgagtgaaaaagtactcaaccaaataataaaacaattgaatttaaaagaaaccgataccgtttacga
aattggaacaggtaaagggcatttaacgacgaaactggctaaaataagtaaacaggtaacgtctattgaattagacagtcat
ctattcaacttatcgtcagaaaaattaaaactgaatactcgtgtcactttaattcaccaagatattctacagtttcaattccctaac
aaacagaggtataaaattgttgggagtattccttaccatttaagcacacaaattattaaaaaagtggtttttgaaagccatgcgt
ctgacatctatctgattgttgaagaaggattctacaagcgtaccttggatattcaccgaacactagggttgctcttgcacactca
agtctcgattcagcaattgcttaagctgccagcggaatgctttcatcctaaaccaaaagtaaacagtgtcttaataaaacttac
ccgccataccacagatgttccagataaatattggaagctatatacgtactttgtttcaaaatgggtcaatcgagaatatcgtcaa
ctgtttactaaaaatcagtttcatcaagcaatgaaacacgccaaagtaaacaatttaagtaccgttacttatgagcaagtattgt
ctatttttaatagttatctattatttaacgggaggaaataattctatgagtcgcttttgtaaatttggaaagttacacgttactaaagg
gaatgtagataaattattaggtatactactgacagcttccaaggagctaaagaggtccctagactctagacccggggatctct
gcagtgagatctggtaatgactctctagcttgaggcatcaaataaaacgaaaggctcagtcgaaagactgggcctttcgtttt
atctgttgtttgtcggtgaacgctctcctgagtaggacaaatccgccgctctagctaagcagaaggccatcctgacggatgg
cctttttgcgtttctacaaactcttgttaactctagagctgcctgccgcgtttcggtgatgaagatcttcccgatgattaattaattc
agaacgctcggttgccgccgggcgttttttatgcagcaatggcaagaacgttgctctagagcggccgcatcgattcacagt
ggcaatctcccccgtattcgtttgaaatgtgccacattaacagcgccgggtgatgtccgtatcgttctgctaataagcggttga
tgtgccgtgttttttctcggtagactttagatgtgaggcagtggttgtgccttccgccgtgcagctgtttgacgcgggaggcatt
gacgcgcaaaacttccggataggtttgcgacagccaggccggacgggctccgctcggcgttgctaatatgacccggccg
cctatactgtgaatccgctcaaaaatatcatccagccattcaaattgatatacgccctcctccggctcaagtgcgctccatgca
aaaatgccgacagaaaacgtattcgtatgagaaagcttcatcagtttgatatcgtcagctaaaatatcgggccgatccagcc
actgatcggggttgtagtctcccccatggagcataaattttgcttttgttacgtgcgttttttcaaccttagacatcaccc.
SEQ ID No. 41
Cttcatataacgcagcatcaatttgttgtgccagatccttgctgaatgaaaaaggcacaatttggtcgtccttagaagc
aatgacagcacggtgcttcgcggactcgatgattttttgatgatcaaacgatccctgtgtaaattcatctaacatctgtaaagtg
ggcaatgatttggcaaagccggaaacaagtatgattccgccaagctgttttcgtaattgaagatgttccaaaaatctcaaaat
ggcggggcaacctaagctgtgcgcaaccaaatacgtattttcatgtaaagtgtgctgatataaagagagtgtatccagccaa
tcctcaagtcttggctggagcggattaggcatgtttaaaatgtctgcttggaccccatctgcgagcagacgttttttcagccaa
gggaaccaatgatttgttgaggaggctctgtagccatgaatgatatagacttgttttgtcattatttcctcctaagaaatattttcat
tcagtataaaattcctctcattgggaaacaagtacgcacttttttgtaagtagctttacatatgaggccctccttgtgccattcccc
acttacacatctcatctatgaggggatacaaagatcgccctctgtcagataacgaatactctactttcagtggggtgtgagaa
acctcagaccgatgaataaggccgtcagtttgcaattccttaagctgtgcgcttaacattttatgggtgatgaagggcatagc
ccttttcagctcgccgtatctcatcgttttttcacaccctaactcatagagaattctcattttccatttgccccctataagagataag
gtgtattcaaatggggcttgtttactgcggcacaggctattcattgtaatcaaacctccttttttaagtatcatatcagaataaatta
ggattttcggtgttgaagtttgagggcgattcacgatctgaagtgaaaggggccgcttgctcaacaggcggcacggtacat
gaatgttctaagtcaaagagggttaccttactgttagttactttatttaaaaaaacgcatgtttttagctgaaaatatctattatggt
atatttaaatataaagaaatatttgagaaatgaggaatggtatgaatacacccaaaaaacataagaaatttaaagcgaaaatg
attctccaaatcatcatggttatcattggaggaatcatagcggcatatggactagaaacagtgctcattccaaacagtgtatct
gatggaggggtgacgggtcttagcgtcgttggttca.
SEQ ID No. 42
Cggtgaaggtctgaatcacagaaataccaagggcgtcaaaacccttggtattttttctggttattagattttatcttcag
ctttacgtgctgcttcgagcttatcaatcctgtctttatatttttctccccagtccttcatgtgcaggataacaggttctaatgttcttc
caaactcagttaaagagtattcgacttttggagggacctgtttataaacctcgcggtgaatcacgccatcccgttccaattctct
taattgtagcgtgagcatacgctgggtaatttttgggtatagttttcgaaactcattaaacctctttttaccgtcaatgagatgata
aagaagaatgcctttccacttacctccgatgatatctaatgttgtctccaccggacatccttcaaagtcatcagaagaataatca
atgccgagtttacgatttctcaagctgttcactcctaatagtatactttttgatactatatacattcaatgtgcgtactttttatatttta
atatacatcatataatgatgtcacatctaaggaatgaaaggaatggatcatatgaaaacactcgtactcgttgtacatccgaat
atagaatcctctcgtatcaataaaaagtggaaagaagccgttttaagtgaaccagatgtaactgtccatgatctttatgaaaaat
atcgcgatcaaccaattgatgtggaatttgaacaacagcagctcctggcccatgaccgtatcgtttttcagtttccattatactg
gtacagcagcccaccgcttttaaaacagtggtttgatgaagtgtttacgtttggctgggctcatggtcccggcggaaataaatt
gaaggggaaagagtgggtaactgccatgtccatcggttcacctgaacactcttatcaagccggcggatataacttgttttcga
taagcgagctgacaaaaccgttccaagcatctgcccatttagtaggcatgacctatcagccttcct.
SEQ ID No. 43
tcttcaactaaagcacccattagttcaacaaacgaaaattggataaagtgggatatttttaaaatatatatttatgttacag
taatattgacttttaaaaaaggattgattctaatgaagaaagcagacaagtaagcctcctaaattcactttagataaaaatttagg
aggcatatcaaatgaactttaataaaattgatttagacaattggaagagaaaagagatatttaatcattatttgaaccaacaaac
gacttttagtataaccacagaaattgatattagtgttttataccgaaacataaaacaagaaggatataaattttaccctgcatttat
tttcttagtgacaagggtgataaactcaaatacagcttttagaactggttacaatagcgacggagagttaggttattgggataa
gttagagccactttatacaatttttgatggtgtatctaaaacattctctggtatttggactcctgtaaagaatgacttcaaagagttt
tatgatttatacctttctgatgtagagaaatataatggttcggggaaattgtttcccaaaacacctatacctgaaaatgctttttctc
tttctattattccatggacttcatttactgggtttaacttaaatatcaataataatagtaattaccttctacccattattacagcagga
aaattcattaataaaggtaattcaatatatttaccgctatctttacaggtacatcattctgtttgtgatggttatcatgcaggattgttt
atgaactctattcaggaattgtcagataggcctaatgactggcttttataatatgagataatgccgactgtactttttacagtcgg
ttttctaatgtcactaacctgccccgttagttgaaggcattttctgtcaatgttttcttacaaagaacgctgtgatatactgaaattt
gtccgtatacattttggaggaatggatatgttaccaaaatacgcgcaagtaaaagaagaaatcagttcttggattaatcaagg
caaaatactgcccgatcaaaaaatccctaccgaaaacgaattaatgcagcaattcggcgtcagccggcataccatccgcaa
agcgatcggagacctcgtatcacaaggtctgctgtacagcgtgcaaggcggaggcacctttgtcgcttcacgctctgctaa
gtcagcgctgcattccaataaaacgatcggtgttttgacaacttacatatcagactatattttcccgagcatcatcagaggaatc
gagtcctatttaagcgagcaggggtattctatgcttttgacaagcacaaacaacaacccggacaatgaaagaagaggctta
gaaaacctgctgtcccagcatattgacggactcatcgtagaaccgacaaaaagcgcccttcaaaccccaaacatcggctat
tatctgaacttggagaaaaacggcattccttttgcgatgattaacgcgtcatatgccgagcttgccgcgccaagttttaccttg
gatgatgtgaaaggcgggatgatggcggcggagcatttgctttctctcggccacacgcatatgatgggtatttttaaagctga
tgacacacaaggcgtgaaacggatgaacggatttatacaggcgcaccgggagcgtgagttgtttccttctccggatatgatc
gtgacatttacaacggaagaaaaagaatcaaaacttctggagaaagtaaaagccacactggagaaaaacagcaagcacat
gccgacagccattctttgttataacgatgaaattgcgctgaaggtgattgatatgctgagggagatggatcttaaagtgccgg
aggatatgtctattgtcgggtacgatgattcacatttcgcccaaatctcagaagtgaaactaacctctgtcaaacatccgaaat
cagtgcttggaaaagcagccgccaaatatgtcattgactgcttagagcataaaaagccgaagcaagaggatgtcatatttga
gcctgagttgatcattcgccagtccgcacgaaaactgaatgaataa.
SEQ ID No. 44
Cggctaacatagaaggagataccctgttaattaccgttgtcggcgggattatccttggattcggaatggggctggcg
ctgcgtaatggcggcgcattagatggaattgacatgcttgctgtgctgctatcccgaaaactgccattcggaacgagcgatc
tcattctattcttaaacctatttgtgtttatttttgtttccacagtatttggcttacaaggggcattgctttcagtcattgcttactacatt
gcatccaaagtgattcatgttgtggaggaaggcttaagcggctctaaaacctttcaaattattacaacccaacctgaattaatg
gtagagacaatacgtgatcaattaggccgaagcgccacatataaagaagcttacgggggtttttcccacgagaaatttaaag
aaatcacatgtgtcatcaaccgtttagaagaaaccaaactaaaggaaattattaatgatattgataaaactgcctttgttaccgt
gtatgatgtagc.
SEQ ID No. 45
gtttatggttgcgttgatggttaaatctcaaaaaggtgttgacattcaaaacaaaatatggtataatgcccccaaaatcg
caaaaaagtgttgacaacttaactcagatctggtataatagaaacacagaatagtcttttaagtaagtctactctgaatttttttaa
aaggagagggtaaagaatggcaaacccaacaacagggaagtcctcgattagggctaagctttctaactcatcgctatcaaa
cctatttaaaaaaaataaaaataaaagacagcgtgaggaaacggaagagcaggacaatgaggataaggatgagagtaag
aaccaggatgaaaataaggacacacagctcactccccgcaagcgtcgccggttgacgaaggagtttgaagagaaggag
gctcgttacaccaacgagttgcccaaggaactgcgcaagtatcgtcctaaaggtttcagattcaatttgcctccaacggatag
acccatcaggatatatgcagatggtgtttttgatcttttccatcttggccacatgaagcaactggaacagtgtaagaaggctttc
cccaatgtaacactgatagttggtgtgcctagcgacaaaatcactcacaaactaaaaggtttgactgtgctgaccgataagc
agcgttgtgaaactttaacgcactgcagatgggttgacgaagtcgtgcccaacgctccctggtgtgtcaccccagaatttcta
ctagaacacaagattgactacgtggcacatgacgatattccttacgttagcgccgacagcgacgatatctacaagccaataa
aggagatgggaaaattcttgactacccaaagaaccaatggtgtctctacaagtgatattatcacaaagatcatcagagattat
gacaaatatttgatgagaaactttgcaaggggtgctaccagacaggagctgaacgtttcttggttgaagaaaaacgaactgg
agttcaaaaaacacatcaatgaattcaggtcatatttcaagaaaaaccagacaaatttgaataacgcctccagagacttgtact
tcgaagtccgtgaaatcttgctaaagaaaacgttgggcaaaaaactctactccaagttaataggcaatgaattaaagaaaca
aaatcaacgacaaagaaaacagaattttttggatgatccgtttactaggaagctaatcagggaggcctctccggctacagag
tttgccaacgaatttacgggcgaaaactctaccgctaaatcaccggatgacaatggaaatcttttcagtcaggaagatgatga
agacacaaattctaacaacacgaatacaaattcagattcagattcaaacactaactcaacgcctcccagtgaagatgacgac
gacaacgacaggttaactttggaaaacctaacacagaagaagaaacaatcagcgaactaagtaaggtcaaaaggaggaa
ttaaaaatggtacaagaatcacgtccagggagtgtaagaagttactcggtcggttaccaagcaaggtccagatcgagttctc
aaagaagacattcgttaacacgccaacgttcctcgcaaagactgattagaaccatcagtatcgagtctgatgtgtctaatatta
ctgacgatgacgatttgagagctgtcaatgagggagtagcgggtgtgcaactggacgtctctgaaaccgcaaataaggga
ccaagaagagcatcagcaactgatgtcacagatagtttgggttcgacttcgtcggaatatattgagattccctttgttaaggaa
acattggatgcaagtttaccttcggattatctgaagcaggacatattaaatctcattcagagtttgaagatatccaaatggtataa
caacaagaaaatccaaccggtagcacaagatatgaacttagtcaagatctctggtgcgatgacaaacgcaattttcaaagtt
gaataccctaagttaccatcgttgctattgagaatatacggaccgaatattgataatatcattgacagggaatatgaattgcag
attttggctaggctttcattgaaaaatataggtccttccctttacggctgttttgtaaacggtagatttgagcagtttctggagaatt
ctaagactttaacaaaagacgacattagaaactggaagaactctcaaaggattgcaaggagaatgaaggagttacatgtag
gtgttcctctcttgagttcagaaaggaagaacgggtcggcttgttggcaaaagattaaccagtggttgcgcacgattgagaa
agtcgaccaatgggtgggggatcctaaaaacattgaaaactctttattatgtgagaattggtccaagtttatggatattgtcgat
agatatcacaagtggcttatttctcaagaacagggtatagagcaagtcaacaaaaatcttatattctgccataatgatgcccaa
tacggcaatttacttttcactgctcctgtgatgaacacaccgagcctatacactgcaccttcgtctacatcattgacttcccaatc
aagttccttatttccttcgagctccaatgtcattgtagatgatataatcaacccgccaaagcaggagcaaagccaagattccaa
attggtcgtcattgattttgaatatgcaggtgccaatcccgccgcatatgatttagcgaatcatctttccgagtggatgtatgatt
acaacaatgctaaggccccacatcagtgccacgctgatagatatcccgataaagaacaggttttgaatttcttatactcttatgt
ttcgcatctaaggggtggtgctaaggaacccatagatgaagaggttcaaagactctataagtcaatcattcaatggagaccc
actgtacaactattttggtcgctctgggccatcctacaaagtggtaaattagagaaaaaagaagcctccactgccatcactag
agaagaaattggacccaatggaaaaaaatatatcatcaagactgaacccgaatcccctgaagaagactttgttgaaaatgac
gacgagcctgaagctggcgtcagcattgacacgttcgattatatggcttatggtcgtgacaagattgcggtcttttggggcga
cctcattggcttaggcataatcaccgaagaagaatgcaaaaatttcagctctttcaagttcctcgatactagttatttgtaatacg
tatacgaattccttcaacaaaggccaaggaaataaagcaaataacaataacaccattattttaattttttttctattactgtcgcta
acatct.
SEQ ID No. 46
cttcatataacgcagcatcaatttgttgtgccagatccttgctgaatgaaaaaggcacaatttggtcgtccttagaagca
atgacagcacggtgcttcgcggactcgatgattttttgatgatcaaacgatccctgtgtaaattcatctaacatctgtaaagtgg
gcaatgatttggcaaagccggaaacaagtatgattccgccaagctgttttcgtaattgaagatgttccaaaaatctcaaaatg
gcggggcaacctaagctgtgcgcaaccaaatacgtattttcatgtaaagtgtgctgatataaagagagtgtatccagccaat
cctcaagtcttggctggagcggattaggcatgtttaaaatgtctgcttggaccccatctgcgagcagacgttttttcagccaag
ggaaccaatgatttgttgaggaggctctgtagccatgaatgatatagacttgttttgtcattatttcctcctaagaaatattttcatt
cagtataaaattcctctcattgggaaacaagtacgcacttttttgtaagtagctttacatatgaggccctccttgtgccattcccc
acttacacatctcatctatgaggggatacaaagatcgccctctgtcagataacgaatactctactttcagtggggtgtgagaa
acctcagaccgatgaataaggccgtcagtttgcaattccttaagctgtgcgcttaacattttatgggtgatgaagggcatagc
ccttttcagctcgccgtatctcatcgttttttcacaccctaactcatagagaattctcattttccatttgccccctataagagataag
gtgtattcaaatggggcttgtttactgcggcacaggctattcattgtaatcaaacctccttttttaagtatcatatcagaataaatta
ggattttcggtgttgaagtttgagggcgattcacgatctgaagtgaaaggggccgcttgctcaacaggcggcacggtacat
gaatgttctaagtcaaagagggttaccttactgttagttactttatttaaaaaaacgcatgtttttagctgaaaatatctattatggt
atatttaaatataaagaaatatttgagaaatgaggaatggtatgaatacacccaaaaaacataagaaatttaaagcgaaaatg
attctccaaatcatcatggttatcattggaggaatcatagcggcatatggactagaaacagtgctcattccaaacagtgtatct
gatggaggggtgacgggtcttagcgtcgttggttcagtttatggttgcgttgatggttaaatctcaaaaaggtgttgacattca
aaacaaaatatggtataatgcccccaaaatcgcaaaaaagtgttgacaacttaactcagatctggtataatagaaacacaga
atagtcttttaagtaagtctactctgaatttttttaaaaggagagggtaaagaatggcaaacccaacaacagggaagtcctcg
attagggctaagctttctaactcatcgctatcaaacctatttaaaaaaaataaaaataaaagacagcgtgaggaaacggaag
agcaggacaatgaggataaggatgagagtaagaaccaggatgaaaataaggacacacagctcactccccgcaagcgtc
gccggttgacgaaggagtttgaagagaaggaggctcgttacaccaacgagttgcccaaggaactgcgcaagtatcgtcct
aaaggtttcagattcaatttgcctccaacggatagacccatcaggatatatgcagatggtgtttttgatcttttccatcttggcca
catgaagcaactggaacagtgtaagaaggctttccccaatgtaacactgatagttggtgtgcctagcgacaaaatcactcac
aaactaaaaggtttgactgtgctgaccgataagcagcgttgtgaaactttaacgcactgcagatgggttgacgaagtcgtgc
ccaacgctccctggtgtgtcaccccagaatttctactagaacacaagattgactacgtggcacatgacgatattccttacgtta
gcgccgacagcgacgatatctacaagccaataaaggagatgggaaaattcttgactacccaaagaaccaatggtgtctcta
caagtgatattatcacaaagatcatcagagattatgacaaatatttgatgagaaactttgcaaggggtgctaccagacaggag
ctgaacgtttcttggttgaagaaaaacgaactggagttcaaaaaacacatcaatgaattcaggtcatatttcaagaaaaacca
gacaaatttgaataacgcctccagagacttgtacttcgaagtccgtgaaatcttgctaaagaaaacgttgggcaaaaaactct
actccaagttaataggcaatgaattaaagaaacaaaatcaacgacaaagaaaacagaattttttggatgatccgtttactagg
aagctaatcagggaggcctctccggctacagagtttgccaacgaatttacgggcgaaaactctaccgctaaatcaccggat
gacaatggaaatcttttcagtcaggaagatgatgaagacacaaattctaacaacacgaatacaaattcagattcagattcaaa
cactaactcaacgcctcccagtgaagatgacgacgacaacgacaggttaactttggaaaacctaacacagaagaagaaac
aatcagcgaactaagtaaggtcaaaaggaggaattaaaaatggtacaagaatcacgtccagggagtgtaagaagttactc
ggtcggttaccaagcaaggtccagatcgagttctcaaagaagacattcgttaacacgccaacgttcctcgcaaagactgatt
agaaccatcagtatcgagtctgatgtgtctaatattactgacgatgacgatttgagagctgtcaatgagggagtagcgggtgt
gcaactggacgtctctgaaaccgcaaataagggaccaagaagagcatcagcaactgatgtcacagatagtttgggttcga
cttcgtcggaatatattgagattccctttgttaaggaaacattggatgcaagtttaccttcggattatctgaagcaggacatatta
aatctcattcagagtttgaagatatccaaatggtataacaacaagaaaatccaaccggtagcacaagatatgaacttagtcaa
gatctctggtgcgatgacaaacgcaattttcaaagttgaataccctaagttaccatcgttgctattgagaatatacggaccgaa
tattgataatatcattgacagggaatatgaattgcagattttggctaggctttcattgaaaaatataggtccttccctttacggctg
ttttgtaaacggtagatttgagcagtttctggagaattctaagactttaacaaaagacgacattagaaactggaagaactctca
aaggattgcaaggagaatgaaggagttacatgtaggtgttcctctcttgagttcagaaaggaagaacgggtcggcttgttgg
caaaagattaaccagtggttgcgcacgattgagaaagtcgaccaatgggtgggggatcctaaaaacattgaaaactctttat
tatgtgagaattggtccaagtttatggatattgtcgatagatatcacaagtggcttatttctcaagaacagggtatagagcaagt
caacaaaaatcttatattctgccataatgatgcccaatacggcaatttacttttcactgctcctgtgatgaacacaccgagccta
tacactgcaccttcgtctacatcattgacttcccaatcaagttccttatttccttcgagctccaatgtcattgtagatgatataatca
acccgccaaagcaggagcaaagccaagattccaaattggtcgtcattgattttgaatatgcaggtgccaatcccgccgcata
tgatttagcgaatcatctttccgagtggatgtatgattacaacaatgctaaggccccacatcagtgccacgctgatagatatcc
cgataaagaacaggttttgaatttcttatactcttatgtttcgcatctaaggggtggtgctaaggaacccatagatgaagaggtt
caaagactctataagtcaatcattcaatggagacccactgtacaactattttggtcgctctgggccatcctacaaagtggtaaa
ttagagaaaaaagaagcctccactgccatcactagagaagaaattggacccaatggaaaaaaatatatcatcaagactgaa
cccgaatcccctgaagaagactttgttgaaaatgacgacgagcctgaagctggcgtcagcattgacacgttcgattatatgg
cttatggtcgtgacaagattgcggtcttttggggcgacctcattggcttaggcataatcaccgaagaagaatgcaaaaatttc
agctctttcaagttcctcgatactagttatttgtaatacgtatacgaattccttcaacaaaggccaaggaaataaagcaaataac
aataacaccattattttaattttttttctattactgtcgctaacatctcggtgaaggtctgaatcacagaaataccaagggcgtcaa
aacccttggtattttttctggttattagattttatcttcagctttacgtgctgcttcgagcttatcaatcctgtctttatatttttctcccc
agtccttcatgtgcaggataacaggttctaatgttcttccaaactcagttaaagagtattcgacttttggagggacctgtttataa
acctcgcggtgaatcacgccatcccgttccaattctcttaattgtagcgtgagcatacgctgggtaatttttgggtatagttttcg
aaactcattaaacctctttttaccgtcaatgagatgataaagaagaatgcctttccacttacctccgatgatatctaatgttgtctc
caccggacatccttcaaagtcatcagaagaataatcaatgccgagtttacgatttctcaagctgttcactcctaatagtatacttt
ttgatactatatacattcaatgtgcgtactttttatattttaatatacatcatataatgatgtcacatctaaggaatgaaaggaatgg
atcatatgaaaacactcgtactcgttgtacatccgaatatagaatcctctcgtatcaataaaaagtggaaagaagccgttttaa
gtgaaccagatgtaactgtccatgatctttatgaaaaatatcgcgatcaaccaattgatgtggaatttgaacaacagcagctcc
tggcccatgaccgtatcgtttttcagtttccattatactggtacagcagcccaccgcttttaaaacagtggtttgatgaagtgttt
acgtttggctgggctcatggtcccggcggaaataaattgaaggggaaagagtgggtaactgccatgtccatcggttcacct
gaacactcttatcaagccggcggatataacttgttttcgataagcgagctgacaaaaccgttccaagcatctgcccatttagta
ggcatgacctatcagccttccttcttcaactaaagcacccattagttcaacaaacgaaaattggataaagtgggatatttttaaa
atatatatttatgttacagtaatattgacttttaaaaaaggattgattctaatgaagaaagcagacaagtaagcctcctaaattca
ctttagataaaaatttaggaggcatatcaaatgaactttaataaaattgatttagacaattggaagagaaaagagatatttaatc
attatttgaaccaacaaacgacttttagtataaccacagaaattgatattagtgttttataccgaaacataaaacaagaaggata
taaattttaccctgcatttattttcttagtgacaagggtgataaactcaaatacagcttttagaactggttacaatagcgacggag
agttaggttattgggataagttagagccactttatacaatttttgatggtgtatctaaaacattctctggtatttggactcctgtaaa
gaatgacttcaaagagttttatgatttatacctttctgatgtagagaaatataatggttcggggaaattgtttcccaaaacacctat
acctgaaaatgctttttctctttctattattccatggacttcatttactgggtttaacttaaatatcaataataatagtaattaccttcta
cccattattacagcaggaaaattcattaataaaggtaattcaatatatttaccgctatctttacaggtacatcattctgtttgtgatg
gttatcatgcaggattgtttatgaactctattcaggaattgtcagataggcctaatgactggcttttataatatgagataatgccg
actgtactttttacagtcggttttctaatgtcactaacctgccccgttagttgaaggcattttctgtcaatgttttcttacaaagaac
gctgtgatatactgaaatttgtccgtatacattttggaggaatggatatgttaccaaaatacgcgcaagtaaaagaagaaatca
gttcttggattaatcaaggcaaaatactgcccgatcaaaaaatccctaccgaaaacgaattaatgcagcaattcggcgtcag
ccggcataccatccgcaaagcgatcggagacctcgtatcacaaggtctgctgtacagcgtgcaaggcggaggcacctttg
tcgcttcacgctctgctaagtcagcgctgcattccaataaaacgatcggtgttttgacaacttacatatcagactatattttcccg
agcatcatcagaggaatcgagtcctatttaagcgagcaggggtattctatgcttttgacaagcacaaacaacaacccggaca
atgaaagaagaggcttagaaaacctgctgtcccagcatattgacggactcatcgtagaaccgacaaaaagcgcccttcaaa
ccccaaacatcggctattatctgaacttggagaaaaacggcattccttttgcgatgattaacgcgtcatatgccgagcttgcc
gcgccaagttttaccttggatgatgtgaaaggcgggatgatggcggcggagcatttgctttctctcggccacacgcatatga
tgggtatttttaaagctgatgacacacaaggcgtgaaacggatgaacggatttatacaggcgcaccgggagcgtgagttgtt
tccttctccggatatgatcgtgacatttacaacggaagaaaaagaatcaaaacttctggagaaagtaaaagccacactggag
aaaaacagcaagcacatgccgacagccattctttgttataacgatgaaattgcgctgaaggtgattgatatgctgagggaga
tggatcttaaagtgccggaggatatgtctattgtcgggtacgatgattcacatttcgcccaaatctcagaagtgaaactaacct
ctgtcaaacatccgaaatcagtgcttggaaaagcagccgccaaatatgtcattgactgcttagagcataaaaagccgaagc
aagaggatgtcatatttgagcctgagttgatcattcgccagtccgcacgaaaactgaatgaataacggctaacatagaagga
gataccctgttaattaccgttgtcggcgggattatccttggattcggaatggggctggcgctgcgtaatggcggcgcattaga
tggaattgacatgcttgctgtgctgctatcccgaaaactgccattcggaacgagcgatctcattctattcttaaacctatttgtgtt
tatttttgtttccacagtatttggcttacaaggggcattgctttcagtcattgcttactacattgcatccaaagtgattcatgttgtgg
aggaaggcttaagcggctctaaaacctttcaaattattacaacccaacctgaattaatggtagagacaatacgtgatcaatta
ggccgaagcgccacatataaagaagcttacgggggtttttcccacgagaaatttaaagaaatcacatgtgtcatcaaccgttt
agaagaaaccaaactaaaggaaattattaatgatattgataaaactgcctttgttaccgtgtatgatgtagc.
SEQ ID No. 47
Gacgataaacccaaacacaacaccaataagaattgtcagcgccctcttattttcaagcggcatcagctgccagcgg
ttggggcgaagttttctatccaaaaacaaacgaacgatgattgaaatggcaaccagcgttaacacatatcctagagcgtgctt
aataatttcttcctgatgctggtgaaaggcggggaacaaatgcaatatgccaatggctagcgaagcactcggaatactgcc
gatggctaagtattttaccaatttaaagttaatcgttttctgccgccaatgggaggcaacgccaaacagctttgtgatcgagtta
tatacaagatccgtaccgaccgcgatggaaggattaattccaagcacaattaaaaggggagttaataaggccgctccaccc
actcccgtcaatccgactaaggctccgacaaacaaacccatcaatatgatgcttacactcatgataaaggcctcctgaaaac
ccgactatttttatcagattatatatgattaggacgcgttaatcaattaaattttttgaatttttgcacgttagttgcttgaaattttggt
atgctcaatatctctattcatttgccctataatgagtgtctggacgaaatcgagaagtactaaaggcgcaaaaaaaagatacct
cttaacaggtatcttttttcttgtttacgctacatgcgctgctgatagaaaggaagctcagtgtggcccatttcccgcacataca
cgaacaggttgcctttatggtgaatttcatgctccattgcaagctgaagcagcgcgcggcctgtgacttttctgccgaaagcg
gatgtcagatcaatttctctgtccagctgctcttcagtcagctgttcaaggatagcgaccgttttttctgtatatgtttttgccagaa
cattgagatctgtttctgtttcttcctgcttgttttgaaatggtgaggcattgccttccttaatgacatttgcgaacaagtggaaag
acgtaagaatgtgtttgaccagctcttctgctgacattgatgtttctgccggtttgtagctgtaatggtctttgctgattttttcggc
aagctcattggtcacgtttcgatgggataaaaaatggctgacaatttgattggattggcacattatgatctcctccatatgtaaa
aataaatggtgcaatgaaatcctatatcaacggttatgaattcacaagtaatatgctgtgggatgaaacaaaatgctatgtcaa
tcgtatatataacgttcacaaaacatgaaatggagtataaacatgactaacatacctttcatttatcagtacgaagaaaaagag
aatgaaagagttgcg.
SEQ ID No. 48
Tcattggcacgaagaagggaatcaccatattgaaaccatggtgaaagacatgacaggagagcaaagaaaaagcc
agcggggaagctggatggaaagaaacaaagtcggttttcactaaaagaaagcacgggtgtttgaaaaacccgtgcttttttg
ttgcggttagccgaaattcgacaattgcggttattttgcgttcttctttttcttgtaaatatgataaaatatgacatatctcgggtaat
tcaaaaggggggattaattgaggatgaagcagacgattccgtcctcttatgtcgggcttaaaattaatgaatggtatactcata
tccggcagttccacgtcgctgaagccgaacgggtcaagctcgaagtagaaagagaaattgaggatatggaagaagacca
agatttgctgctgtattattctttaatggagttcaggcaccgtgtcatgctggattacattaagccttttggagaggacacgtcgc
agctagagttttcagaattgttagaagacatcgaagggaatcagtacaagctgacagggcttctcgaatattactttaattttttt
cgaggaatgtatgaatttaagcagaagatgtttgtcagtgccatgatgtattataaacgggcagaaaagaatcttgccctcgt
ctcggatgatattgagaaagcagagtttgcttttaaaatggctgagattttttacaatttaaaacaaacctatgtttcgatgagcta
cgccgttcaggcattagaaacataccaaatgtatgaaacgtacaccgtccgcagaatc.
SEQ ID No. 49
Ggttgattggagcctgagcatgtggcatatatatttgaaaagcttgaaaagaaaacctttgcttacgatgaagatgag
aaagaaccggattataccgtccataagtctataagaaacagtgtatacgcgtatcctgaaaagggagttgcttttgccagaat
tccatattttcaagatgggagcattatgagttttgattgtctgtttgcggttaacgatgaaaagatgcgtgcatttctggagggag
tcagaccgcgtctttgggaaaaaagcaagcggaaagtgaccgtgtttacggatggagatggagggacttcaagagagca
ggaagccattgtcagagaggttcagcggagtcaagtcatcatgaatccgctattgaaaaaagagatatacagatcaattgat
cagttttttcatagtgataaatcgttttatcaaacatatgacatcccttacaagcgcggcattctgttatatggacctcctggaaac
ggaaagacgacgttagtgaagtcgatcgcaggcagtatcgatgcacctgttgcttattggcaaattactgaatttacgtcgag
cgagacaatagaagaagtctttcaggcagcgagacgcctcgctcctgcagttctggtcatcgaggatatagattcgatgcc
ggaagatgtgcggtccttttttctcaatacgcaggacg.
SEQ ID No. 50
gtttatggttgcgttgatggttaaatctcaaaaaggtgttgacattcaaaacaaaatatggtataatgcccccaaaatcg
caaaaaagtgttgacaacttaactcagatctggtataatagaaacacagaatagtcttttaagtaagtctactctgaatttttttaa
aaggagagggtaaagaatggcaaacccaacaacagggaagtcctcgattagggctaagctttctaactcatcgctatcaaa
cctatttaaaaaaaataaaaataaaagacagcgtgaggaaacggaagagcaggacaatgaggataaggatgagagtaag
aaccaggatgaaaataaggacacacagctcactccccgcaagcgtcgccggttgacgaaggagtttgaagagaaggag
gctcgttacaccaacgagttgcccaaggaactgcgcaagtatcgtcctaaaggtttcagattcaatttgcctccaacggatag
acccatcaggatatatgcagatggtgtttttgatcttttccatcttggccacatgaagcaactggaacagtgtaagaaggctttc
cccaatgtaacactgatagttggtgtgcctagcgacaaaatcactcacaaactaaaaggtttgactgtgctgaccgataagc
agcgttgtgaaactttaacgcactgcagatgggttgacgaagtcgtgcccaacgctccctggtgtgtcaccccagaatttcta
ctagaacacaagattgactacgtggcacatgacgatattccttacgttagcgccgacagcgacgatatctacaagccaataa
aggagatgggaaaattcttgactacccaaagaaccaatggtgtctctacaagtgatattatcacaaagatcatcagagattat
gacaaatatttgatgagaaactttgcaaggggtgctaccagacaggagctgaacgtttcttggttgaagaaaaacgaactgg
agttcaaaaaacacatcaatgaattcaggtcatatttcaagaaaaaccagacaaatttgaataacgcctccagagacttgtact
tcgaagtccgtgaaatcttgctaaagaaaacgttgggcaaaaaactctactccaagttaataggcaatgaattaaagaaaca
aaatcaacgacaaagaaaacagaattttttggatgatccgtttactaggaagctaatcagggaggcctctccggctacagag
tttgccaacgaatttacgggcgaaaactctaccgctaaatcaccggatgacaatggaaatcttttcagtcaggaagatgatga
agacacaaattctaacaacacgaatacaaattcagattcagattcaaacactaactcaacgcctcccagtgaagatgacgac
gacaacgacaggttaactttggaaaacctaacacagaagaagaaacaatcagcgaactaagtaaggtcaaaaggaggaa
ttaaaaatggtacaagaatcacgtccagggagtgtaagaagttactcggtcggttaccaagcaaggtccagatcgagttctc
aaagaagacattcgttaacacgccaacgttcctcgcaaagactgattagaaccatcagtatcgagtctgatgtgtctaatatta
ctgacgatgacgatttgagagctgtcaatgagggagtagcgggtgtgcaactggacgtctctgaaaccgcaaataaggga
ccaagaagagcatcagcaactgatgtcacagatagtttgggttcgacttcgtcggaatatattgagattccctttgttaaggaa
acattggatgcaagtttaccttcggattatctgaagcaggacatattaaatctcattcagagtttgaagatatccaaatggtataa
caacaagaaaatccaaccggtagcacaagatatgaacttagtcaagatctctggtgcgatgacaaacgcaattttcaaagtt
gaataccctaagttaccatcgttgctattgagaatatacggaccgaatattgataatatcattgacagggaatatgaattgcag
attttggctaggctttcattgaaaaatataggtccttccctttacggctgttttgtaaacggtagatttgagcagtttctggagaatt
ctaagactttaacaaaagacgacattagaaactggaagaactctcaaaggattgcaaggagaatgaaggagttacatgtag
gtgttcctctcttgagttcagaaaggaagaacgggtcggcttgttggcaaaagattaaccagtggttgcgcacgattgagaa
agtcgaccaatgggtgggggatcctaaaaacattgaaaactctttattatgtgagaattggtccaagtttatggatattgtcgat
agatatcacaagtggcttatttctcaagaacagggtatagagcaagtcaacaaaaatcttatattctgccataatgatgcccaa
tacggcaatttacttttcactgctcctgtgatgaacacaccgagcctatacactgcaccttcgtctacatcattgacttcccaatc
aagttccttatttccttcgagctccaatgtcattgtagatgatataatcaacccgccaaagcaggagcaaagccaagattccaa
attggtcgtcattgattttgaatatgcaggtgccaatcccgccgcatatgatttagcgaatcatctttccgagtggatgtatgatt
acaacaatgctaaggccccacatcagtgccacgctgatagatatcccgataaagaacaggttttgaatttcttatactcttatgt
ttcgcatctaaggggtggtgctaaggaacccatagatgaagaggttcaaagactctataagtcaatcattcaatggagaccc
actgtacaactattttggtcgctctgggccatcctacaaagtggtaaattagagaaaaaagaagcctccactgccatcactag
agaagaaattggacccaatggaaaaaaatatatcatcaagactgaacccgaatcccctgaagaagactttgttgaaaatgac
gacgagcctgaagctggcgtcagcattgacacgttcgattatatggcttatggtcgtgacaagattgcggtcttttggggcga
cctcattggcttaggcataatcaccgaagaagaatgcaaaaatttcagctctttcaagttcctcgatactagttatttgtaatacg
tatacgaattccttcaacaaaggccaaggaaataaagcaaataacaataacaccattattttaattttttttctattactgtggcta
actcca.
SEQ ID No. 51
tcttcctgatgctggtgaaaggcggggaacaaatgcaatatgccaatggctagcgaagcactcggaatactgccga
tggctaagtattttaccaatttaaagttaatcgttttctgccgccaatgggaggcaacgccaaacagctttgtgatcgagttatat
acaagatccgtaccgaccgcgatggaaggattaattccaagcacaattaaaaggggagttaataaggccgctccacccac
tcccgtcaatccgactaaggctccgacaaacaaacccatcaatatgatgcttacactcatgataaaggcctcctgaaaaccc
gactatttttatcagattatatatgattaggacgcgttaatcaattaaattttttgaatttttgcacgttagttgcttgaaattttggtat
gctcaatatctctattcatttgccctataatgagtgtctggacgaaatcgagaagtactaaaggcgcaaaaaaaagatacctct
taacaggtatcttttttcttgtttacgctacatgcgctgctgatagaaaggaagctcagtgtggcccatttcccgcacatacacg
aacaggttgcctttatggtgaatttcatgctccattgcaagctgaagcagcgcgcggcctgtgacttttctgccgaaagcgga
tgtcagatcaatttctctgtccagctgctcttcagtcagctgttcaaggatagcgaccgttttttctgtatatgtttttgccagaaca
ttgagatctgtttctgtttcttcctgcttgttttgaaatggtgaggcattgccttccttaatgacatttgcgaacaagtggaaagac
gtaagaatgtgtttgaccagctcttctgctgacattgatgtttctgccggtttgtagctgtaatggtctttgctgattttttcggcaa
gctcattggtcacgtttcgatgggataaaaaatggctgacaatttgattggattggcacattatgatctcctccatatgtaaaaat
aaatggtgcaatgaaatcctatatcaacggttatgaattcacaagtaatatgctgtgggatgaaacaaaatgctatgtcaatcg
tatatataacgttcacaaaacatgaaatggagtataaacatgactaacatacctttcatttatcagtacgaagaaaaagagaat
gaaagagttgcggtttatggttgcgttgatggttaaatctcaaaaaggtgttgacattcaaaacaaaatatggtataatgcccc
caaaatcgcaaaaaagtgttgacaacttaactcagatctggtataatagaaacacagaatagtcttttaagtaagtctactctg
aatttttttaaaaggagagggtaaagaatggcaaacccaacaacagggaagtcctcgattagggctaagctttctaactcatc
gctatcaaacctatttaaaaaaaataaaaataaaagacagcgtgaggaaacggaagagcaggacaatgaggataaggatg
agagtaagaaccaggatgaaaataaggacacacagctcactccccgcaagcgtcgccggttgacgaaggagtttgaaga
gaaggaggctcgttacaccaacgagttgcccaaggaactgcgcaagtatcgtcctaaaggtttcagattcaatttgcctcca
acggatagacccatcaggatatatgcagatggtgtttttgatcttttccatcttggccacatgaagcaactggaacagtgtaag
aaggctttccccaatgtaacactgatagttggtgtgcctagcgacaaaatcactcacaaactaaaaggtttgactgtgctgac
cgataagcagcgttgtgaaactttaacgcactgcagatgggttgacgaagtcgtgcccaacgctccctggtgtgtcacccc
agaatttctactagaacacaagattgactacgtggcacatgacgatattccttacgttagcgccgacagcgacgatatctaca
agccaataaaggagatgggaaaattcttgactacccaaagaaccaatggtgtctctacaagtgatattatcacaaagatcatc
agagattatgacaaatatttgatgagaaactttgcaaggggtgctaccagacaggagctgaacgtttcttggttgaagaaaaa
cgaactggagttcaaaaaacacatcaatgaattcaggtcatatttcaagaaaaaccagacaaatttgaataacgcctccaga
gacttgtacttcgaagtccgtgaaatcttgctaaagaaaacgttgggcaaaaaactctactccaagttaataggcaatgaatta
aagaaacaaaatcaacgacaaagaaaacagaattttttggatgatccgtttactaggaagctaatcagggaggcctctccgg
ctacagagtttgccaacgaatttacgggcgaaaactctaccgctaaatcaccggatgacaatggaaatcttttcagtcagga
agatgatgaagacacaaattctaacaacacgaatacaaattcagattcagattcaaacactaactcaacgcctcccagtgaa
gatgacgacgacaacgacaggttaactttggaaaacctaacacagaagaagaaacaatcagcgaactaagtaaggtcaa
aaggaggaattaaaaatggtacaagaatcacgtccagggagtgtaagaagttactcggtcggttaccaagcaaggtccag
atcgagttctcaaagaagacattcgttaacacgccaacgttcctcgcaaagactgattagaaccatcagtatcgagtctgatg
tgtctaatattactgacgatgacgatttgagagctgtcaatgagggagtagcgggtgtgcaactggacgtctctgaaaccgc
aaataagggaccaagaagagcatcagcaactgatgtcacagatagtttgggttcgacttcgtcggaatatattgagattccct
ttgttaaggaaacattggatgcaagtttaccttcggattatctgaagcaggacatattaaatctcattcagagtttgaagatatcc
aaatggtataacaacaagaaaatccaaccggtagcacaagatatgaacttagtcaagatctctggtgcgatgacaaacgca
attttcaaagttgaataccctaagttaccatcgttgctattgagaatatacggaccgaatattgataatatcattgacagggaata
tgaattgcagattttggctaggctttcattgaaaaatataggtccttccctttacggctgttttgtaaacggtagatttgagcagttt
ctggagaattctaagactttaacaaaagacgacattagaaactggaagaactctcaaaggattgcaaggagaatgaaggag
ttacatgtaggtgttcctctcttgagttcagaaaggaagaacgggtcggcttgttggcaaaagattaaccagtggttgcgcac
gattgagaaagtcgaccaatggggggggatcctaaaaacattgaaaactctttattatgtgagaattggtccaagtttatgga
tattgtcgatagatatcacaagtggcttatttctcaagaacagggtatagagcaagtcaacaaaaatcttatattctgccataat
gatgcccaatacggcaatttacttttcactgctcctgtgatgaacacaccgagcctatacactgcaccttcgtctacatcattga
cttcccaatcaagttccttatttccttcgagctccaatgtcattgtagatgatataatcaacccgccaaagcaggagcaaagcc
aagattccaaattggtcgtcattgattttgaatatgcaggtgccaatcccgccgcatatgatttagcgaatcatctttccgagtg
gatgtatgattacaacaatgctaaggccccacatcagtgccacgctgatagatatcccgataaagaacaggttttgaatttctt
atactcttatgtttcgcatctaaggggtggtgctaaggaacccatagatgaagaggttcaaagactctataagtcaatcattca
atggagacccactgtacaactattttggtcgctctgggccatcctacaaagtggtaaattagagaaaaaagaagcctccact
gccatcactagagaagaaattggacccaatggaaaaaaatatatcatcaagactgaacccgaatcccctgaagaagacttt
gttgaaaatgacgacgagcctgaagctggcgtcagcattgacacgttcgattatatggcttatggtcgtgacaagattgcggt
cttttggggcgacctcattggcttaggcataatcaccgaagaagaatgcaaaaatttcagctctttcaagttcctcgatactagt
tatttgtaatacgtatacgaattccttcaacaaaggccaaggaaataaagcaaataacaataacaccattattttaattttttttcta
ttactgtggctaactccatcattggcacgaagaagggaatcaccatattgaaaccatggtgaaagacatgacaggagagca
aagaaaaagccagcggggaagctggatggaaagaaacaaagtcggttttcactaaaagaaagcacgggtgtttgaaaaa
cccgtgcttttttgttgcggttagccgaaattcgacaattgcggttattttgcgttcttctttttcttgtaaatatgataaaatatgaca
tatctcgggtaattcaaaaggggggattaattgaggatgaagcagacgattccgtcctcttatgtcgggcttaaaattaatgaa
tggtatactcatatccggcagttccacgtcgctgaagccgaacgggtcaagctcgaagtagaaagagaaattgaggatatg
gaagaagaccaagatttgctgctgtattattctttaatggagttcaggcaccgtgtcatgctggattacattaagccttttggag
aggacacgtcgcagctagagttttcagaattgttagaagacatcgaagggaatcagtacaagctgacagggcttctcgaat
attactttaatttttttcgaggaatgtatgaatttaagcagaagatgtttgtcagtgccatgatgtattataaacgggcagaaaag
aatcttgccctcgtctcggatgatattgagaaagcagagtttgcttttaaaatggctgagattttttacaatttaaaacaaacctat
gtttcgatgagctacgccgttcaggcattagaaacataccaaatgtatgaaacgtacaccgtccgcagaatctcttcaactaa
agcacccattagttcaacaaacgaaaattggataaagtgggatatttttaaaatatatatttatgttacagtaatattgacttttaaa
aaaggattgattctaatgaagaaagcagacaagtaagcctcctaaattcactttagataaaaatttaggaggcatatcaaatg
aactttaataaaattgatttagacaattggaagagaaaagagatatttaatcattatttgaaccaacaaacgacttttagtataac
cacagaaattgatattagtgttttataccgaaacataaaacaagaaggatataaattttaccctgcatttattttcttagtgacaag
ggtgataaactcaaatacagcttttagaactggttacaatagcgacggagagttaggttattgggataagttagagccacttta
tacaatttttgatggtgtatctaaaacattctctggtatttggactcctgtaaagaatgacttcaaagagttttatgatttatacctttc
tgatgtagagaaatataatggttcggggaaattgtttcccaaaacacctatacctgaaaatgctttttctctttctattattccatgg
acttcatttactgggtttaacttaaatatcaataataatagtaattaccttctacccattattacagcaggaaaattcattaataaag
gtaattcaatatatttaccgctatctttacaggtacatcattctgtttgtgatggttatcatgcaggattgtttatgaactctattcag
gaattgtcagataggcctaatgactggcttttataatatgagataatgccgactgtactttttacagtcggttttctaatgtcacta
acctgccccgttagttgaaggcattttctgtcaatgttttcttacaaagaacgctgtgatatactgaaatttgtccgtatacattttg
gaggaatggatatgttaccaaaatacgcgcaagtaaaagaagaaatcagttcttggattaatcaaggcaaaatactgcccga
tcaaaaaatccctaccgaaaacgaattaatgcagcaattcggcgtcagccggcataccatccgcaaagcgatcggagacc
tcgtatcacaaggtctgctgtacagcgtgcaaggcggaggcacctttgtcgcttcacgctctgctaagtcagcgctgcattcc
aataaaacgatcggtgttttgacaacttacatatcagactatattttcccgagcatcatcagaggaatcgagtcctatttaagcg
agcaggggtattctatgcttttgacaagcacaaacaacaacccggacaatgaaagaagaggcttagaaaacctgctgtccc
agcatattgacggactcatcgtagaaccgacaaaaagcgcccttcaaaccccaaacatcggctattatctgaacttggagaa
aaacggcattccttttgcgatgattaacgcgtcatatgccgagcttgccgcgccaagttttaccttggatgatgtgaaaggcg
ggatgatggcggcggagcatttgctttctctcggccacacgcatatgatgggtatttttaaagctgatgacacacaaggcgtg
aaacggatgaacggatttatacaggcgcaccgggagcgtgagttgtttccttctccggatatgatcgtgacatttacaacgg
aagaaaaagaatcaaaacttctggagaaagtaaaagccacactggagaaaaacagcaagcacatgccgacagccattctt
tgttataacgatgaaattgcgctgaaggtgattgatatgctgagggagatggatcttaaagtgccggaggatatgtctattgtc
gggtacgatgattcacatttcgcccaaatctcagaagtgaaactaacctctgtcaaacatccgaaatcagtgcttggaaaagc
agccgccaaatatgtcattgactgcttagagcataaaaagccgaagcaagaggatgtcatatttgagcctgagttgatcattc
gccagtccgcacgaaaactgaatgaataaggttgattggagcctgagcatgtggcatatatatttgaaaagcttgaaaagaa
aacctttgcttacgatgaagatgagaaagaaccggattataccgtccataagtctataagaaacagtgtatacgcgtatcctg
aaaagggagttgcttttgccagaattccatattttcaagatgggagcattatgagttttgattgtctgtttgcggttaacgatgaa
aagatgcgtgcatttctggagggagtcagaccgcgtctttgggaaaaaagcaagcggaaagtgaccgtgtttacggatgg
agatggagggacttcaagagagcaggaagccattgtcagagaggttcagcggagtcaagtcatcatgaatccgctattga
aaaaagagatatacagatcaattgatcagttttttcatagtgataaatcgttttatcaaacatatgacatcccttacaagcgcggc
attctgttatatggacctcctggaaacggaaagacgacgttagtgaagtcgatcgcaggcagtatcgatgcacctgttgctta
ttggcaaattactgaatttacgtcgagcgagacaatagaagaagtctttcaggcagcgagacgcctcgctcctgcagttctg
gtcatcgaggatatagattcgatgccggaagatgtgcggtccttttttctcaatacgcaggacg.

What is claimed is:

1. A recombinant engineered strain for de novo synthesis of CDP-choline using glucose as a substrate, wherein the recombinant engineered strain is obtained by modifying Bacillus subtilis 168N as a chassis as follows:
(1) modification of Bacillus subtilis 168N: integrating the phosphatidylethanolamine N-methyltransferase gene PEM1 and phosphatidylethanolamine/phosphatidyl-N-methylethanolamine N-methyltransferase gene PEM2 from Saccharomyces cerevisiae into an artificial operon P_{xyl}-PEM2-PEM1, and integrating the artificial operon P_{xyl}-PEM2-PEM1 into the lacA site of the Bacillus subtilis 168N genome for induced expression, wherein the Bacillus subtilis 168N is obtained by integrating Pₐᵣₐ-neo into the araR site of the wild-type Bacillus subtilis 168 genome;
(2) construction of recombinant engineered strain: integrating the artificial operon TP2-CCT-CKI into the genome of Bacillus subtilis expressing P_{xyl}-PEM2-PEM2-PEM1 in step (1), thus constructing a recombinant engineered strain capable of de novo synthesis of CDP-choline using glucose as a substrate;
the artificial operon TP2-CCT-CKI is composed of the choline kinase gene CKI and the phosphocholine cytidylyltransferase gene CCT as well as the constitutive strong promoter TP2.

2. The application of the recombinant engineered strain according to claim 1 in de novo synthesis of CDP-choline using glucose as the substrate.

3. The application according to claim 2, wherein the specific application comprises inoculating the single colony of the recombinant engineered strain into a test tube filled with LB liquid culture medium, conducting shake culture, then transferring the inoculum volume of 0.5% to 2% of the fermentation medium volume to a conical flask containing the fermentation medium, conducting shake culture, and then adding xylose with a final concentration of 10 g/L after 2-5 hours of fermentation.

4. The application according to claim 3, wherein the LB liquid culture medium comprises the following components: tryptone 5-15 g/L, yeast extract 1-9 g/L, NaCl 5-15 g/L, erythromycin 1-8 μg/mL, neomycin 10-20 μg/mL.

5. The application according to claim 3, wherein the fermentation medium comprises the following components: glucose 20-80 g/L, tryptone 5-15 g/L, yeast extract 1-9 g/L NaCl 5-15 g/L, MgSO₄·7H₂O 0.02-3 g/L, cytidine-5′-phosphate 0.01-4 g/L, and xylose 5-20 g/L;
the fermentation conditions are: pH 6.0-8.0, fermentation temperature 30-45° C., rotation speed 100-250 r/min, fermentation time 20-50 h.

6. A method for de novo synthesis of phosphatidylcholine using glucose as a substrate, comprising the following steps:
(1) modification of Bacillus subtilis 168N: combining the phosphatidylethanolamine N-methyltransferase gene PEM1 and phosphatidylethanolamine/phosphatidyl-N-methylethanolamine N-methyltransferase gene PEM2 from Saccharomyces cerevisiae into an artificial operon P_{xyl}-PEM2-PEM1, and integrating the artificial operon P_{xyl}-PEM2-PEM1 into the lacA site of the Bacillus subtilis 168N genome for induced expression;
(2) construction of recombinant engineered strain: integrating the artificial operon TP2-CCT-CKI into the genome of Bacillus subtilis expressing P_{xyl-}PEM2-PEM2-PEM1 in step (1), thus constructing a recombinant engineered strain capable of de novo synthesis of CDP-choline using glucose as a substrate;
(3) inoculating the single colony of the recombinant engineered strain constructed in step (2) into a test tube filled with LB liquid culture medium, conducting shake culture, then transferring the inoculum volume of 0.5% to 2% of the fermentation medium volume to a conical flask containing the fermentation medium, then adding xylose with a final concentration of 10 g/L after 2-5 hours of fermentation, and then conducting shake culture in the conical flask for 20-50 hours to synthesize CDP-choline.
